(19) United States
(12) Patent Application Publication
(10) Pub. No.: US 2004/0138156 A1
(43) Pub. Date: Jul. 15, 2004

(54) Therapeutic regulation of deoxyribonuclease-1-like-3 activity

(76) Inventors: Michael C. Schneider, Springfield, IL (US); Andrew Wilber, Springfield, IL (US)

Correspondence Address: FITCH EVEN TABIN AND FLANNERY, 120 SOUTH LA SALLE STREET, SUITE 1600, CHICAGO, IL, 60603-3406, US

(21) Appl. No.: 10/378,098

(22) Filed: Feb. 26, 2003

Related U.S. Application Data
(60) Provisional application No. 60/359,619, filed on Feb. 26, 2002.

(51) Int. Cl. A61K 48/00; A61K 9/127; C12N 15/88; A61K 38/21

(52) U.S. Cl. 514/44; 435/458; 424/450; 424/85.6

(57) ABSTRACT

Deoxyribonuclase 1-like 3 (D3) hydrolyzes lipid-complexed DNA and decreases transfection efficiency in liposomal transfection (lipofection) Systems. Accordingly, D1L3 provides a more accurate test of the efficiency of lipid/liposomal based gene therapy than current standards using deoxyribonuclease 1 (D1). Moreover, it has been found that mice with systemic lupus erythematosus (lupus) have lowered D1L3 activity. Therefore, differing therapeutic benefits may result from either the upward or downward therapeutic regulation of D1L3 activity. For example, blocking D1L3 activity enhances liposomal transfection for gene therapy, while increasing D1L3 activity may enhance destruction of pathogenic DNA, whether viral, bacterial or endogenous. Destruction of pathogenic DNA may provide treatment for lupus, or viral and oncogenic diseases.

### RELATED APPLICATION

Pursuant to 35 U.S.C. § 119(e), this application is based on, and claims the benefit of, U.S. Provisional Application Serial No. 60/359,619, filed on Feb. 26, 2002, which is hereby incorporated by reference in its entirety.

### FIELD OF THE INVENTION

Deoxyribonuclease 1-like 3 (D1L3 or D3) hydrolyzes DNA. The enzyme, when added to the media bathing cultured cells, blocks or decreases liposomal gene transfection (lipofection) efficiency; in other words it confers a barrier to liposomal gene transfection (henceforth this is also called BT, or the function will referred to as BT activity or BT effect). This enzyme's BT activity has varied but significant potency against a variety of cationic lipid transfection reagents. In addition, the scope of this effect may not only apply to lipofection reagents since D1L3 is also shown to be active in vitro in blocking adenoviral infection (FIG. 30).

Liposomal gene therapy in vitro is highly efficient, but there are significant barriers to in vivo efficiency. These experiments predict that macrophage-secreted D1L3 activity is likely one of the main tissue and serum barriers for liposomal transfection in vivo. This would predict that blocking or decreasing such activity, directly or indirectly, would enhance liposomal transfection as used in in vivo gene therapy, while increasing D1L3 activity may enhance destruction of pathogenic DNA, whether viral, bacterial or endogenous, thereby providing treatment for lupus, or viral and oncogenic diseases.

Serum contains at least two deoxyribonucleases (DNASES): DNASE1L3 and DNASE I. The latter very likely contributes towards most of the serum DNASE activity against free DNA. The former enzyme can be detected in serum by immunoblot, and likely forms part of the actin-resistant DNASE component. The presence of this enzyme in serum suggests that it could play a role in the in vivo barriers to liposomal gene transfection in many tissues.

Systemic lupus erythematosus (SLE or lupus) is an autoimmune disorder characterized by the presence of autoantibodies against nuclear components, such as DNA and chromatin. Macrophages from a model of murine SLE, pre-symptomatic NZB/W F1, cannot provide HeLa cells with a barrier to liposomal transfection, while BT activity is present in media conditioned by non-lupus prone C57BL mice. In addition, evaluation of DNASE1L3 sequences in African American patients with SLE identifies a patient heterozygous for a complete loss of function mutation in this gene; and many patients with an intronic mutation (IVS6+5G>T) that is predicted to affect splicing efficiency, hence as a hypomorphic allele, lead to decreased DNASE1L3 activity. This predicts the polygenic human SLE will also be characterized by an absence of a macrophage-secreted barrier activity. Sequencing assays and/or assays of DNASE1L3 activity can be used as a diagnostic test for predisposition to disease, and to identify a target activity that could either decrease the risk or progression to SLE.

### BACKGROUND

Relevance of Discovery to Liposomal Gene Transduction and Viral Infection. Significant research today is directed towards improving the ability to transduce exogenous genetic material into cells or tissues for treatment of diseases. Once a therapeutically effective gene is engineered into an expression vector, its efficacy still depends on the ability to productively incorporate the engineered gene into the patient's cells in the tissues. Viral transduction and lipofection are the two most commonly used methods of in vivo gene therapy in trials; other methods include electroporation, non-lipid based transduction, and particle transfer (e.g., a gene gun). At present, of over 600 human gene therapy trials (http://www.wiley.co.uk/genetherapy/clinical/), about 70% of the trials utilized viral delivery systems and 13% use liposomal gene techniques.

Lipofection was first coined for the use of synthetic cationic lipids, which form unilamellar liposomes in transfection (Feigner et al., (1987) Proc. Natl. Acad. Sci. USA 84, 7413-7). Liposome self-assembly appears to occur via electrostatic interaction between the lipids and anionic DNA. Newer liposome preparations may include monocationic lipids, polycationic lipids, or neutral lipids, including cholesterol, gangliosidosis, PEG-polymerized lipids, and fusogenic compounds to enhance stability or uptake by cells (Gao et al., (1995) Gene. Ther. 2, 710-22). For example, FuGENE™-6, a highly efficient, proprietary, non-liposomal lipid preparation, is available from Roche Diagnostics Corp. (Indianapolis, Ind.).

Liposomal transfection is a complex process. Typically lipid-linked polycationic moieties condense around a negatively charged DNA core and form lamellar-surrounded vesicular structures. These vesicles appear to enter cells easily and abundantly via endocytosis (Gao et al., (1995) Gene. Ther. 2, 710-22; Godbey et al., (1999) Proc. Natl. Acad. Sci. USA 96, 5177-81). The rate-limiting step for nuclear localization in vitro appears to be the exodus of the DNA from the endosomes and into the cytoplasm (Xu et al., (1996) Biochemistry 35, 5616-23; Lechardeur et al., (1999) Gene. Ther. 6, 482-97; Zabner et al., (1995) J. Biol. Chem. 270, 18997-9007).

The efficiency of lipofection in vivo is lower than in vivo viral transduction (infection) with retrovirus, adenovirus, or adeno-associated virus (Crystal, (1995) Science 270, 404-10). This is not surprising, since viruses are selected by evolution for successful replication. Thus, viral transduction, which is highly efficient, has become the method of choice for delivering genetic material into an organism. However, viral transduction is prone, not unexpectedly, to induce greater inflammation than non-viral gene transduction. Accordingly, a need exists to improve the efficiency of lipofection as a viable alternative to viral transduction. In some ways the goal of gene therapy vector development is to design carrier agents that possess the evolutionarily refined efficiency of viral agents with the simplicity and non-inflammatory characteristics of liposomal reagents. Part of the answer lies in deciphering the pathways and mechanisms by which virus-derived gene therapy reagents evade the barriers to gene expression and efficiently introduce their material into cells. In addition, there is a need to decipher the tissue and cellular barriers to liposomal transfection.

The barriers to liposomal and viral gene transfection are roughly understood. Cells from the reticuloendothelial lineage (for example, tissue macrophages) play a role in scavenging of transduced DNA (Takakura et al., (1999) Pharm. Res. 16, 503-8; Takagi et al., (1998) Biochem. Biophys. Res. Commun. 245, 729-33). Cellular and serum nucleases also play a role, though nearly all the attention has focused on DNASE I or D1 and the non-homologous lysosomal enzyme “acid” DNASE II, which is active in acidic milieu in the absence of divalent cations (Barry et al., (1999) Hum. Gene. Ther. 10, 2461-80; Ross et al., (1998) Gene. Ther. 5, 1244-50).

Deoxyribonucleases (DNases or also called DNAases) are enzymes that catalyze the hydrolysis and breakdown of polymeric strands of deoxyribonucleic acid. They can be subdivided, for example, into endo- and exo-nucleases, or site-specific endonucleases. This invention will henceforth refer to endonucleases homologous to DNASE I (or DNASE1 or DNase I or D1, the product of the HUMDNASEI locus, accession # AAA63170; also accession M55983.1) as DNASEs. The only exception to this nomenclature in eukaryotes is the existence of a DNASE II (or DNASE2, the product of DRN2_HUMAN locus, accession # 000115); this is a non-homologous acid DNASE. The main focus of this invention is an activity of a member of the DNASE1 gene family (DNASE1L3, or DNase-1-like-3, or D3—the product in humans of the DNASE1L3 locus on 3p21 (accession # NM_{—}004944) and in mouse of the syntenic DNASE1L3 locus on chromosome 14 (sequence is represented by the locus and accession # NM_{—}007870, and in rats by locus and accession # NM_{—}053907, U75689 (DNASE gamma), or AF039852 (DNase Y); and in Xenopus by accession # AF059612). The experiments contained herein mainly utilize the murine sequences, but are almost certainly applicable to all homologous mammalian DNASE1L3s, which are highly conserved.

In humans, there are at least 4 DNASE loci: DNASE I, DNASE1 L1, DNASE1 L2, and DNASE1L3 (FIG. 1). They are named as homologues (“DNASE1-like” enzymes) of the cardinal member of the family, DNASE1. All share similar core nuclease domains and structure, requiring divalent cations (calcium and magnesium) for activity and inhibited by zinc. Unlike DNASE 2, a lysosomal endonuclease active at acidic pH, DNASES are active at neutral pH (Shiokawa et al., (2001) Biochemistry 40, 143-52). All are predicted to derive from an ancestral gene, and in fact, domains exist with affinities to bacterial enzymes such as exonuclease III, suggesting a superfamily of nuclease proteins. In humans, the distinct DNASE loci encode proteins with generally distinct tissue expression patterns, thus likely non-redundant functions in vivo. This is also supported by the observation that DNASE1L3, unlike DNASE I and DNASE1L2, has a distinctive and longer C-terminal extension.

It has been postulated that one or more of these extra- and/or intracellular nucleases are responsible for the barriers to efficient liposomal transfection of DNA. Because most current studies attribute serum DNase activity to D1, most studies of non-viral DNA transfection (e.g., lipofection) efficiency utilize D1 as the standard barrier (Xu et al., (1996) Biochemistry 35, 5616-23; Crook et al., (1998) Gene. Ther. 5, 137-43; Mullen et al., (2000) Biochim. Biophys. Acta 1523, 103-10; Niidome et al., (1997) J. Biol. Chem. 272, 15307-12). However, in vitro studies reveal that D1 has little effect on transfection efficiency. Accordingly, a need exists to provide an assay to investigate the DNASE that more accurately reflects the barrier to liposomal gene transfection, and thus define agents that may better evade such a barrier.

Relevance of Discovery to Systemic Lupus Erythematosus (SLE).

These observations are relevant to systemic lupus erythematosus (SLE or lupus), an autoimmune disorder characterized by autoantibodies against nuclear components, such as DNA and chromatin. It has been hypothesized that predisposition to SLE includes defects in the clearance of DNA-associated antigens. These antigens trigger the autoimmune response. The importance of antigens in SLE pathogenesis is supported by the affinity maturation of anti-DNA antibodies, a cardinal feature of the disease (Shlomchik et al., (1990) J. Exp. Med. 171, 265-92; Burlingame et al., (1994) J. Clin. Invest. 94, 184-92; Mohan et al., (1993) J. Exp. Med. 177, 1367-81). Affinity maturation shows that as the disease progresses, continued exposure to DNA-allied antigens, selects for cells expressing autoantibodies of higher affinity. The observation that Anti-DNA antibodies show this progressive increase in high-affinity mutations strongly suggests that antigen drives in large part the disease. The present model is that DNASEs are pivotal in this process of degradation, and that deficiency of this process, specifically D1L3 activity, predisposes to SLE susceptibility.

The defects in the innate immunity system seen in SLE strongly imply that inability to clear specific antigen or antigens bound in immune-complexes, underlies SLE. In many previous models of SLE, the broad diversity of antigenic targets suggested no unifying pathogenic antigens. The experiments do not clearly fully predict the character and components of the antigenic material, other than it is associated with DNA, and in fact the material may in fact be a constellation of complex debris that includes intracellular nuclear elements, DNA, and phospholipid membranes, all targets found in the SLE autoantibody repertoire, and likely all targets exposed, for example, in apoptotic debris. DNASEs are not predicted to be the sole determinants of the degradation of these complex antigens, but certainly one of them. Chromosomal DNA, by virtue of its size, represent a formidable molecule, which may make the degradation of other associated elements more difficult. DNA may be of endogenous or exogenous (infectious) origin.

SLE pathogenesis may be subdivided into different stages, where different predispositions play a role. For example, while the pathways leading to the original dysregulation and production of pathogenic autoantibodies or immune complexes may be shared by large number of patients, the organ distribution and nature of complications, that is terminal manifestations, may be highly individualized. These “inciting” mechanisms may not be identical in all patients but are likely to have some shared features. For example, all patients with lupus may have a defect, either secondary or primary in some arm of the antigen clearance system, for example complement (possibly C1, C4, or C2) or surface or serum proteins associating with chromatin, such as serum amyloid-P (SAP) (Bickerstaff et al., (1999) Nat. Med. 5, 694-7) or scavenging receptors (Takagi et al., (1998) Biochem. Biophys. Res. Commun. 245, 729-33), or finally one of the DNASEs, either DNASE1 or DNASE1L3. They may in addition or else have defects governing the general activation or responsiveness of leukocytes, including possibly suppressor and helper T-cell subsets, dendritic cells, and B-cells. These defects may affect, among others, cytokines, cytokine receptors, transcriptional modifiers, or intracellular messengers. Finally the inciting factors may include non-genetic modifiers, for example lectins or drug exposures (Miyasaka, (1996) Intern. Med. 35, 527-8) or sex-limited modifiers, or genetic modifiers that are sex-limited. The model for a polygenic disease is that each individual inherits or encounters a set of predispositions in overlapping or distinct categories. DNASE1L3 deficiency is one of the predispositions, and perhaps a common one, since there may be primary defects in the enzyme (suggested by the 89I mutation in NZ and MRL strains) and secondary defects, such as a failure of Ifn-γ induced macrophage BT activity.

Deficiencies of DNASE1 have long been postulated to predispose to SLE (Fros et al., (1968) Clin. Exp. Imm. 3, 447-455) by interfering with the clearance or processing of DNA-associated antigens (Walport, (2000) Nat. Genet. 25, 135-6). In this model, nuclear material is viewed antigenic debris, perhaps mostly the residua from apoptosis that requires clearance by a janitorial DNASE function. The characteristic presence of the anti-DNA and anti-nucleosomal antibodies suggested that DNA-associated antigens might be hastening disease development or progression, and that, correspondingly, antigen degradation could hamper this predisposition. DNASE I has long been identified and partially purified from bovine sources, and was predicted to be present in serum, thus this enzyme was the initial and prime candidate for the nuclease defective in SLE. Serum DNASE activity by RDA has been reported to be low in NZB/W (Macanovic et al., (1997) Clin. Exp. Immunol. 108, 220-6) and human SLE (Chitrabamrung et al., (1981) Rheumatol. Int. 1, 55-60; Tew et al., (2001) Arthritis Rheum. 44, 2446-7), and almost absent from the D1 −/− mice (Napirei et al., (2000) Nat. Genet. 25, 177-81); these studies attribute this serum activity solely to the D1 enzyme. Deoxyribonuclease 1 (D1) −/− mice have been found to develop a phenotype similar to SLE (Napirei et al., (2000) Nat. Genet. 25, 177-81). Recently, a null allele in D1 has been found in two Japanese pedigrees with SLE associated with elevated IgG anti-nucleosomal titers (Yasutomo et al., (2001) Nat. Genet. 28, 313-4). The findings to date do suggest that DNASE1 deficiency can exacerbate or predispose to SLE; yet, D1 defects are unusual in human and murine SLE (Tew et al., (2001) Arthritis Rheum. 44, 2446-7). In addition, therapeutic trials with D1 in NZ mice (Macanovic et al., (1996) Clin. Exp. Immunol. 106, 243-52; Verthelyi et al., (1998) Lupus 7, 223-30) and a human trial with human DNase I, albeit phase I and well-tolerated, (Davis et al., (1999) Lupus 8, 68-76) had mixed, if any, success in ameliorating the progression of disease.

The role of DNASE1 deficiency in human or murine polygenic SLE and its potential for SLE protection appear limited. The enzyme may contribute to a portion of SLE-protection. DNASE I appears to target endocytosed DNA heading towards lysosomes by virtue of its glycosylation. DNA-associated material entering the cell by other pathways may to have more relevance to antigenic detection. The non-overlapping functions of D1 and D1L3 may explain why D1 alone does not suffice for SLE protection nor is an effective treatment for lupus. The data presented shows an Ifn-γ inducible BT activity is secreted by bone-marrow derived macrophages, and this activity correlates in normal macrophages with D1L3 levels and DNASE activity. The in vitro experiments establish that D1L3 conditioned media can convey BT activity, and suggest macrophage-conditioned BT activity is equivalent to D1L3 activity. Finally, a defect in BT activity is present in macrophages from the NZB/W F1 mouse, which has both a D1L3 missense mutation and defects in macrophage-secreted DNASE, also has deficient BT activity. Hence a defect in BT activity, primary or secondary, and almost certainly due to D1L3 deficiency, commonly underlies polygenic SLE.

In a broader perspective, a biologic role for D1L3 is to protect of cell nuclei and other intracellular compartments from exogenous DNA. DNA-containing apoptotic debris may resemble to liposomal particles containing DNA. Macrophage D1L3 likely helps degrade these apoptotic remnants in vivo and preventing their conversion, in an SLE-susceptible immune system, into inciting autoantigens. In addition, D1L3 may be protecting cellular genomes from “autotransfection”, which could lead to oncogenesis (Holmgren et al., (1999) Blood 93, 3956-63; Bergsmedh et al., (2001) Proc. Natl. Acad. Sci. USA 98, 6407-11). D1L3 may cooperate with other agent of innate immunity, such as complement, to clear of DNA-associated material. Finally, it is likely aiding in the protection against viral infection. In this invention, an effect of D1L3 on adenoviral infection is demonstrated. This activity is predicted to possibly have wider applications in antiviral (for example, both DNA and RNA viruses including hepatitis viruses, herpes viruses, adeno-associated viruses, and retroviruses) or anti-microbial therapy for intracellular pathogens, including Listeria, mycobacteria, and others.

### SUMMARY OF THE INVENTION

The present invention provides a method to test the efficiency of liposomal reagents in vitro and their resistance against a composition comprising D1L3 added to cell culture media or supernatant. The success of a liposomal composition can be measured by transduction efficiency following exposure of a transfection composition to a target cell in the presence of D 1 L3.

The present invention also provides a method of protecting against, treating, or reversing the progression of lupus in a mammal, said method comprising increasing D1L3 activity in the mammal. The invention describes an abnormality of D1L3 in a polygenic model of murine lupus and explains how this could exacerbate the phenotype.

The present invention also provides a method of destroying a pathogenic encapsulated membrane bound or micelle-bound DNA comprising increasing D1L3 activity. This would include endogenous and non-endogenous infectious particles containing DNA, and even possibly RNA. The material can be present as non-expressible or expressible endogenous chromatin, or engineered DNA constructs, or pathogenic viral, rickettsial, mycobacterial, mycoplasma, yeast, or bacterial genomes.

The present invention also provides a method of treating a disease in a mammal by gene therapy, said method comprising the administration of a gene therapy composition to the mammal, wherein the gene therapy composition comprises a recombinant gene to affect the gene therapy, a lipofection reagent, and a D1L3-activity-reducing agent.

The present invention provides a gene therapy composition suitable for use in mammals, including humans, comprising a recombinant gene to affect the gene therapy, a lipofection reagent, and a D3-activity-reducing agent. Suitable lipofection reagents include monocationic lipids, polycationic lipids, DEAE, dextran, lipo-polyamines, and cholesterol. Suitable D3-activity-reducing agents include antibodies, peptides, DNA fragments, and chemicals, which reduces or moderates D1L3 activity in vivo. More specifically, such D3-activity-reducing agents selectively inhibits D1L3 expression, inhibits D1L3 nuclease activity, inhibits C-terminus activity, complexes D1L3, and/or degrades D1L3.

The utility of such an agent that inhibits BT activity is apparent from the present discovery; such agents would enhance gene therapy using either liposomal or adenoviral or other vectors. Agents that might be of utility in this matter include:

(1) Peptide reagents that bind D3 and inhibit its activity, identified through combinatorial libraries or other methods.

(2) Monoclonal or other antibodies that bind D1L3 protein and inhibit its BT activity, either directly or by interfering with its biologic cellular enhancement of BT activity.

(3) Chemicals based on reagents which are able to block the activity of the enzyme in vitro, including zinc and chelating agents such as EDTA.

Lower D1L3 activity may predispose to polygenic SLE. Increasing D1L3 levels may benefit SLE patients. A partial loss of function mutation (T891) is common to two independent mice models of polygenic lupus (the NZB/W hybrid and the MRL strain).

Using GFP-tagged adenovirus methods parallel to liposomal studies, D1L3 and D1-D3 CT were shown to afford a barrier to infection, i.e., block in vitro adenoviral infection of HeLa cells (FIG. 30). This demonstrates that, in addition to blocking transfection of liposome bound DNA, these enzymes may also block viral DNA. This also implicates other pathogenic viruses, such as retroviruses (e.g., HIV), hepadnaviruses (HBV), hepatitis virus (e.g., Hepatitis C), small pox, measles and herpes virus and are believed to provide a novel, broad-based in vivo antiviral activity. Thus, increasing D1L3 expression may also useful in resisting viral or oncogenic diseases, by targeting nucleosomal DNA. Further, the ability of these enzymes to block human adenoviral infection may result in methods to make gene therapy more efficient and less toxic. For example, anti-D3 antibodies or D1L3 inhibitors may enhance adenoviral transfection.

Recombinant human D1L3 can also synthesize by baculovirus or eukaryotic cells. Such D1L3 is administered intravenously to augment native D1L3 that is already in the serum. To increase D1L3 or D1+D3 CT activity, intravenous infusion or expression can lead to increased tissue and/or circulating levels of these enzymes in an effective amount to prevent or treat viral infections.

Media conditioned with Ifn-γ treated macrophages from the C57BL strain also blocks lipofection. This is not surprising, since it is known that Ifn-γ induces macrophage secretion of D1L3 and increases D1L3 activity.

Thus, in vivo activation of D1L3 activity may be accomplished by administering effective amounts of Ifn-γ to induce expression of D1L3 by macrophages. It is also known that lipopolysaccharides (LPS), phrobol myristate acetate (PMA), and Ifn-γ (all macrophage activators) also induce D1L3 levels.

### BRIEF DESCRIPTION OF THE FIGURES

FIG. 1. Alignment of DNASE I family of proteins. Panel A: Alignment of human DNase 1L3 and other DNase family members. The alignment demonstrates conserved motifs and residues in the core nuclease domains, while there is divergence at C-termini. Panel B: Summary of information for the 4 DNASEs.

FIG. 2. Nothern analysis of DNASE1L3 expression in normal tissues.

Methods described in Rodriguez et al. ((1997) Genomics 42, 507-13).

Human cDNA probe was used to probe multiple tissue northerns from Clontech. Note highly specific expression in liver.

FIG. 3. Alignment of C-termini of DNASE1L3s from various species with both D1 and D1L1. Alignment begins at highly conserved SDH motif. All D1L3-CTs have a predominance of basic residues, even Xenopus D1L3 with a shorter C-terminal extension.

FIG. 4. Depiction of BT assay.

FIG. 5. Comparison of Nuclease activity. D1-contains more nuclease activity against free DNA than D3-media. Panel A: RDA results show representative nuclease activities of equivalent volumes of media conditioned by cells transfected with 2 mg of either vector pcDNA, D1, D3, D3DCT, or N196K-D3. Nearly no clearing is visible in control and N196K-D3 media, while D1 and D3DCT-media consistently showed greater RDA activity than D3. Panel B: Zymogram of equivalent volumes (5 ml/lane) of pcDNA-, D1-, D3-media, and serum show the broader band of activity produced by media containing glycosylated D1 (33-36 kDA) versus the smaller sharper 28-29 kDa band of D3 activity. Serum zymograms appear to show a doublet of activities; migration of serum proteins may be altered relative to media due to differences in albumin concentration. Panel C: Parallel SDS-PAGE lanes of murine serum (5 ml) used for zymogram (left) and anti-DNASE1L3 peptide immunoblot demonstrate the presence of D3 in the serum, and the possible relationship to serum zymogram activities; however, it is not clear if the visible serum nuclease bands reflect D3.

FIG. 6. D3-media confers a barrier to liposomal transfection (BT). Panel A: Western of D3-transfected cells. Anti-D3 immunoblots show similar levels of protein in cell lysates from D3, D3DCT, and N196K-D3 expressing cells. This expression level is derived for the cells conditioning media in FIG. 6B. Panel B: Western of GFP-transfected cells. Anti-GFP immunoblots at 1:5000 (Clontech) of transfected HeLa cells demonstrate that D3-media prevents GFP expression, presumably by blocking gene transduction. Expression of GFP in D1, D3DCT, and N196K-D3 treated cells did not differ significantly from controls. Immunoblots using anti-b-actin demonstrate near equal loading of cell-lysates.

FIG. 7. Fluorescent microscopy of GFP activity in cells. GFP fluorescence can be seen by microscopy in cells exposed to D1-media but not D3-media. Hela cells transfected with n1-eGFP in the presence of conditioned media with DNASE1 (D1-CM) or DNASE1L3 (D3-CM) show that GFP expression by fluorescent microscopy only in cells exposed to DNASE1. HeLa cells are rinsed in PBS, then fixed in 4% paraformaldehyde dissolved in PBS for 30 minutes, then resuspended in PBS and photographed through a FITC high power scope.

FIG. 8. Comparison of RDA-DNASE activity and GFP-transfection efficiency. The brown columns represent RDA-DNASE units per 5 ml conditioned media for D3DCT (n=16), D1 (n=24), and D1D3CT (n=4) media. Mean standard deviations are noted. Both N196K-D3 (n=2) and control media (n=24) had similar negligible background RDA activity. Green columns represent the mean ratio of GFP plasmid transfection efficiency (as deduced from GFP expression measured by anti-GFP western blot) relative to that seen for parallel control transfection (i.e., pCDNA) for D3DCT (n=9), N196K-D3 (n=2), D1 (n=20), and D1D3CT (n=4). D3- and D1D3CT-media respectfully abolished or significantly reduced transfection efficiency in exposed cells. Despite greater RDA-nuclease activity for D3DCT and D1-media, there was no significant decrease in transfection efficiency relative to control-media.

FIG. 9. BT activity of diluted D3-media. Immunoblots of transfected HeLa cells demonstrate that D3-media and ten-fold dilutions appear to impair transfection of plasmid. No GFP expression is clearly detectable in ten fold dilutions of D3 media. Similar results were obtained with dilutions of D3 with D3-N196K. Ratio of GFP expression per lane relative to control are depicted in the lower register.

FIG. 10. Dilutions of D3-media maintain BT activity. Immunoblots of GFP transfected HeLa cells demonstrate that D3-media when diluted ten fold with media conditioned by N196K-D1L3 transduced HeLa cells has BT activity. No GFP expression is clearly detectable in five and ten fold dilutions of D1L3 media. Lane 1 (control); lane 2 (N196K); lane 3 D1L3-CM, lane 4-5 are 1:5 and 1:10 dilutions of D1L3 with N196K media. Actin controls in lower register are from the GFP-transduced cells. The addition of N196K at ten-fold dilution does not allow for transduction, suggesting there is no dominant negative effect of an inactive protein.

FIG. 11. BT activity occurs with multiple liposomal reagents. Panel A: The following figures show anti-GFP immunoblots of GFP-transduced cells in a variety of conditioned media. The secondary GFP transduction (as opposed to the DNASE-transduction) is carried out in the labeled liposomal reagent. Panel B: Scope of BT effect on various reagents. Table summarizes the results to date; however this table is not meant to be the definitive description of the resistance or sensitivity of the BT activity, but implicates the activity as present with a variety of reagents.

FIG. 12. Murine mutagenesis primers for N196K primers and human D1L3 N191K. Panel A: The N196K-D3 cDNA clone was created by point mutagenesis of the murine clone using primer GGTGATTTCAAgGCCGGCTGTAGCTA 3′ and its complement. The mutation was confirmed by the creation of a novel Hae3 site in the cDNA. The encoded change is bolded below with the primer under the mismatched sequence. Panel B: A similar experiment using N191K (human homologous mutation), wherein the GST fusion is shown to have no activity. First four lanes are anti-D3 immunoblot with lane 1 control, lane 2 GST cDNA, lane 3 N191K shows expected 56 kDa band just as lane 4 which is wild-type human GST fusion. In lane 5-7 are the zymographic results of cell lysates of E. Coli induced to express GEX fusions with lane 5 (no insert), lane 6 N191K-humanD3, and lane 7 wild type GST-human D3. No activity is seen in N191K lane. Western analysis performed using antisera #497 at 1:4000. In preliminary experiments, addition of ZnCl2 to induction media at a concentration 2 mM, enhances the levels of the 56 kDa nuclease.

FIG. 13. Mutagenesis of pcDNA-D1L3 to create D3DCT. Using the forward primer depicted under the nucleotide sequence, the wild type murine D1L3 was mutagenized to create a stop codon (TAA) at codon 289.

FIG. 14. Creation of chimeric fusion of N-terminal D1 to C-terminal D1L3 (289-end). Panel A: Sequence of MusD1 F128. Panel B: Sequence of fFusion protein D1+D3CT. Panel C: Sequence of fusion cDNA clone D1+D3CT. To synthesize D1D3CT, the murine DNASE1 cDNA was obtained as follows: (1) Mus musculus mRNA for deoxyribonuclease I, sequence locus D83038, accession # D83038 with superimposed primers (F128 and R1029) used for RT-PCR (reverse transcription followed by PCR). The resulting clone encoding the full-length enzyme was cloned into pcDNA3.1, then mutagenized with the primer DNASE1-BGL2F: CACTCAGAAAgATCTGATGTCATTG and its reverse complement. Stop codon is underlined in previous forward primer. Primer creates a Bgl2 site just upstream of stop codon. This step was followed by the silent mutagenesis of the remaining single Bgl2 site in the vector pcDNA 3.1 (data not shown). The C-terminus of D1L3 (cloned previously in pBluescript plasmid) was amplified with M13F and the D3-CT-Bam Forward primer GCGGATCCGGGCCTTCACCAACAACAGAA 3′. This fragment was cloned as a BamH1-KPN1 cut fragment into BGL2-KPN cut pcDNA3.1(-Bgl2) containing DNASE1 [+Bgl2]. The resulting fusion sequence depicted in Panel B with DNASE I encoded amino acids in small case while D1L3 is in capital letters. The resulting chimeric cDNA is depicted in Panel C with DNASE1 in small case unbolded, fusion link marked with slash mark, DNASE1L3 CT capitalized, and a 5 base linker sequence (gatcc) in bold letters.

FIG. 15. D1+D3CT enzyme has BT activity. Panel A: Top portion provides a representative RDA activity from one plate for the labeled clone. Bottom portion provides a GFP immunoblot of HeLa lysates that demonstrate gene transduction levels using FuGene-6 in control, D1, D3, and D1+D3CT-media. Mean ratio of transfection efficiency and activity of D1+D3CT is graphically depicted in FIG. 8. Panel B: Two putative N-linked glycosylation sites present in two mammalian enzymes (mouse top and bovine bottom) (Nishikawa et al., (1999) J. Biol. Chem. 274, 19309-15). N-linked glycosylation sites in bovine and murine highlighted. Panel C: Sequence for the mutagenesis primers used to sequentially alter the N-linked glycosylation sites. The asparagine codon underlined in each foward primer. Panel D: Zymograms of media conditioned by HeLa cells transfected with pcDNA-expressing clones (from left to right): (1) pcDNA; (2) DNASE1; (3) D1-N40S; (4) D1-N128S; (5) D1-(N40S+N128S) clone #1; (6) D1-(N40S+N128S) clone #2; (7) D1+D3CT; (8) D1+D3CT-N40S; (9) D1+D3CT-N128S; (10) D1+D3CT-(N40S+N128S) clone #1; (11) D1+D3CT-(N40S+N128S) clone #2. The (N40S+N128S) clones are double mutants with both asparagines altered. On the left are DNASE1-derived clones, those on right half are D1+D3CT-derived. As is evident, loss of glycosylation leads to smaller, sharper bands of activity. Bottom part of the figure are the GFP immunoblot of HeLa Cell lysates that were exposed to the media conditioned by the clones directly above in the zymogram. Hence, pcDNA (control)-conditioned media does not establish a barrier to liposomal transfection (lane 1); however, the loss of glycosylation sites enhances the BT activity of both D1 and D1+D3CT clones against FuGene-6 transfected N1-eGFP. Also notable is that despite lower levels of activity on zymogram and RDA (data not shown) the de-glycosylated D1+D3CT show complete BT activity, while the glycosylated D1+D3CT still allows some transfection to occur.

FIG. 16. DNASE-conditioned media do not fully degrade Fugene-coated plasmid. Representative experiment (n=3) of exposure of 2 mg free (−) or liposomal-bound (+FuGene) plasmid to control media with 10% fetal calf serum (lanes 1-2), D1 (lanes 3-4), D1L3 (lanes 5-6), D3DCT (lanes 7-8), and N196K-D3 (lanes 9-10).

FIG. 17. Sequence and alignment of mutant and normal DNASEs. Panel A: Wild-type (top) and mutant 89I (lower) sequence traces from C57BL and MRL respectively. Arrows point to where 89I mutation is caused by a T to C transition at base pair 438. Panel B demonstrates that ATA to ACA codon change due to the transition at bp 438 substitutes isoleucine for threonine in the protein. Panel C illustrates where the amino acid change occurs relative to other D1L3 (rat and human) and bovine DNASE 1. Also depicted are residues in DNASE I, that are thought by crystallography to be involved in actin and DNA binding respectfully. Full alignments of DNASEs can be found in Rodriguez et al. ((1997) Genomics 42, 507-13).

FIG. 18. Genotyping of murine strains. Panel A: The PCR genotyping strategy to determine which strains are 89T and which are 89I is depicted; it uses mismatched reverse primer to detect mutant amplimers as SSP1-digestible (AATATT), while the 48 bp fragment amplified from wild-type strains is SSP1-resistant. Results from genotyping are arranged below each strain showing both original PCR (−) and after addition of SSP1 (+) amplimer products. Both C57BL and BxSB strains contain the wild-type allele, while all other mice depicted are 89I, hence SSP1 digestible. The forward genotyping primer was F413-musD3 5′-AATGGAAATTCACGAAGAAGCAC, while the mismatched primer R461-musD3SSP was 5′-CGAGAACTAATCACATAGTTGaAT, where the non-capitalized a is a mismatched base pair. Panel B illustrates use of the strategy to evaluate further strains. The same strategy was used on the BXD strains, whose parents are discrepant, to map the position of D1L3 to its expected syntenic position on murine 14 by comparing the transmission to the markers referred to in the text. For analysis 3 ul of 25 ul PCR reaction digested with Ssp I for 1 hour at 37° C. and run a 8% acrylamide/1× TAE gel w/o EtBr at 100 volts. Stained in EtBr bath 10 mg/mL.

FIG. 19. Comparisons of 89I and 89T enzymes. Rows A and B represent two representative experiments of immunoblots and zymograms of D3-GST fusions expressed in E. coli, and purified by GST-columns. In row A, anti-D3 immunoblots performed on column-purified ^{˜}56 kDa GST-fusions; while row B represents the parallel DNA-SDS-zymograms performed on equal loads of the respective sample. While protein levels are nearly equal (p<0.01) between 89I and 89T, the 89I enzyme. Rows C and D represent two representative experiments of immunoblots of cell lysates and zymograms of conditioned media for D3 enzymes expressed by transduced HeLa. Row C provides a representative experiment where the anti-D3 immunoblot detecting the ^{˜}29 kDa D1L3 in cell lysates of HeLa cells transfected with pcDNA3.1-D3; while row D provides the DNA-SDS-zymograms performed on equal loads of the respective conditioned media. This demonstrates the ^{˜}50% decreased activity found in the 89I enzyme relative to protein levels.

FIG. 20. Graph of nuclease activity comparisons of 89T- and 89I-GST-D3 and pcDNA-D3-enzymes. Bars represent the pixel volumes (x 103) for the normalized mean nuclease activities on zymograms for both the GST-fusions expressed in bacteria, then GST-column purified, and of the HeLa-expressed enzymes. The pixel volumes (area×intensity) are normalized relative to levels of protein. The observed nuclease activity is versus free DNA. This graph summarizes the data from the experiments depicted in FIG. 19. The table summarizes the mean normalized activity values and standard deviation.

FIG. 21. Anti-D1L3 western of normal mouse tissues. (A) Polyclonal anti-D3 immune western blot of murine whole organ protein lysates, (b) control pre-immune western blot, and (c) added peptide blocked westernblot. Two bands of activity in the liver sample are thought to reflect cleaved and uncleaved enzyme (34 kDa); while the ^{˜}29 kDa bands in spleen and thymus reflects cleaved enzyme. Anti-mus DNASE1L3 peptide rabbit antisera, detects expected the 34 and 29 kDa proteins in murine spleen, liver, thymus and faintly in lung. Negative pre-immune and peptide blocking controls shown. Primary antibody used at 1:5000 in powdered milk with 0.05% Tween and 1% goat serum; HRP-linked 2° antibodies at 1:4000 (Amersham ECL), and exposed on radiographs. To derive protein extracts, whole organs were homogenized and boiled in 2% SDS-PAGE loading buffer without DTT. Protein load per lane equalized by Bradford assay, confirmed by visual inspection of Ponceau Red stained Immobilon blots (Biorad mini-Protean II apparatus). Negative lanes of brain, heart, small gut and kidney are not shown.

FIG. 22. Normal and induced expression of D1L3 in murine macrophages. This figure demonstrates the immunological detection of DNASE1L3 by anti-D1L3 peptide immunoblot of cell lysates from macrophages or adherent splenocytes presumed to be of macrophage lineage. In anti-D1L3 blots of cell lysates, the protein is seen both as a ^{˜}34 kDa uncleaved molecule and a ^{˜}28 kDa cleaved molecule. In addition the figures demonstrate higher levels of expression of D1L3 in bone marrow macrophages from MRL, NZB, and BXSB mice compared to C57BL mice. Finally, the figure shows that the D1L3 levels in macrophages are inducible by Ifn-γ in C57BL, as well as RDA nuclease activity. This was shown to be correlated with increased BT activity. Panel A: Demonstrating an anti-D1L3 immunoblot of cell lystates from murine splenocytes. Cells were plated on serum-coated tissue culture dishes and incubated overnight, the next morning, non-adherent cells (N) were rinsed vigorously with PBS 1×, and both adeherent (A) and non-adherent cells (N) from C57BL, NZB/W, BXSB, and MRL were lysed and immunoblotted with anti-D1L3. While the ^{˜}34 kDa size band consistent with uncleaved D1L3 is present in both adherent and non-adherent, the cleaved form (^{˜}28 kDa) is present almost exclusively in the adherent population of presumed macrophages. Also noticable are the higher levels of expression of D1L3 in the 3 latter lupus strains. Panel B: Untreated bone marrow macrophages from NZB/WF1 mice have a much higher baseline expression expression of D1L3 than C57BL macrophages.

However, treatment of the cells with 100 U/ml of Ifn-γ leads to significant induction of D1L3 levels. Induction is present in macrophages from both strain though the absolute level of induction appears greater in C57BL than in the SLE-prone NZB/W F1 strain. NZB/W protein loads in Ifn-γ-negative lane is slightly underloaded relative to actin. Panel C: Bone marrow macrophages isolated from bone marrow (via adherence) from MRL and C57BL mice, and cultured for 2 days in vitro, were induced by Ifn-γ. In the top register, an anti-D1L3 immunoblot demonstrates induction of D1L3 expression as both 34 and 28 kDa fractions. More significantly, no DNASE activity is detected by zymogram pre-induction, but a 28 kDa band is detectable after induction with interferon gamma. The level of RDA nuclease activity is also increased after interferon gamma induction in both MRL and B6 mice. No induction of D1L3, RDA-DNASE, or zymographic DNASE activity is evident upon tratement with 100 U/ml of Ifn-alpha. Bottom register shows actin levels in macrophage cell lysates by immunoblot in order to demonstrate equal loading. Panel D: Bone marrow macrophages from C57BL and MRL mice cultured for 7 days in the presence of GM-CSF were induced with Ifn-γ, again showing the higher baseline levels of 28 kDa enzyme in the lupus prone strain, and in C57BL showing induction of levels aftern Ifn-γ. The baseline level of D1L3 expression in C57BL is higher, and the degree of induction is lower than that observed in macrophages grown in vitro for shorter culture times (<48 hours) in the absence of GM-CSF. Panel E: HeLa cells exposed to conditioned media from HeLa cells 100 U/ml of Ifn-γ do not have a barrier to liposomal transfection. HeLa cells treated with the same dose of Ifn-γ did not have BT activity either (data not shown).

FIG. 23. Table of RDA-DNASE and D1L3 levels in SLE and C57BL mice. Table shows pixel values (volumes =area×intensity) of D3 levels by anti-D1L3 immuno and nuclease activity by RDA, along with standard errors and significance. Measurements were performed on C57BL, BXSB, NZB/W, and MRL mice. Often, more than one RDA assay on a specific sample were conducted. The mean and standard error of measurements is cited. All RDA assays were incubated for an equivalent time under the same conditions. All immunoblots were performed under similar conditions. No D1L3 immunoblot was performed in urine. D1L3 levels and DNASE activity in samples from three lupus models (BXSB, NZB/W F1, and MRL) and one control strain (C57BL) are shown. All values are expressed in pixels. All scans were analyzed by Scion Image using 250 pixels per inch, 100% size image. The most notable results are the high levels of D1L3 protein induction in C57BL splenocytes and bone-marrow derived macrophages, despite absence of parallel induction of DNASE activity. In addition, both NZ and MRL mice had significantly less activity in macrophage conditioned media than C57BL. See text for isolation and culture conditions.

FIG. 24. Zymograms and RDAs of murine urine. This figure shows similar zymographic (Panel A) and urine RDA-DNase (Panel B) levels for NZB/W, MRL, BXSB, and C57BL mice.

FIG. 25. Zymograms and anti-D3 westerns of murine sera. Panel A: Top row depicts anti-DNAS1L3 western of sera from various murine strains. The second row, Zymograms of same samples, which shows not only the decreased activity of nucleases in 89I sera, but also the loss of the upper band of DNASE activity. The bottom row depicts a representative RDA for the samples. 3 mice were bled 4 times each over a course of 4 days. Data represented in table form in FIGS. 23 and 28. Panel B: Parallel zymogram of normal serum and DNASE-transfected HeLa cells demonstrates the relationship of the serum activities to D1 and D1L3, and suggests that most of the serum DNASE activity is D1.

FIG. 26. Anti-D3 westerns of splenocyte cell lysates and RDAs of splenocyte-conditioned media. Equivalent numbers and concentrations of splenocytes (counted with hemochromocytometer and incubating both adherent and non-adherent) were cultured for 24-36 hours to condition media. Panel is representative of experiments using 3 mice for each strain: the register in row a is a western using anti-D3 peptide antisera. The western demonstrates the induced level of D1L3 in splenocytes from all lupus-prone strains. Row b provides an immunoblot detecting b-actin expression, which is used as a control for levels of cellular protein. In row c, intraexperimental (same plate) RDAS of conditioned media are reproduced for conditioned media samples. The results indicate that despite the induced levels of cellular D1L3, there is no parallel increase of RDA nuclease activity. Mean values were derived for all three experiments by pixel analysis of scanned images and represented in table II and FIG. 6. The differences in levels of protein expression were significant for expression levels between C57BL and all lupus mice (P<0.05), while DNASE activity was not significant.

FIG. 27. Anti-D3 westerns of macrophage cell lysates and RDAs of macrophage-conditioned media. Macrophages were derived from bone marrow. Row a is representative of experiments using 3 mice for each strain: the register in row a is a western using anti-D3 peptide antisera. The western demonstrates the induced level of D1L3 in macrophages from lupus-prone strains. Row b provides an immunoblot detecting b-actin expression, which is used as a control for levels of cellular protein. In row c, intraexperimental (same plate) RDAS of conditioned media are reproduced for conditioned media samples. The results indicate that despite the induced levels of cellular D1L3, there is no parallel increase of RDA nuclease activity. Mean values were derived for expression and activity by pixel analysis of scanned images and are represented in FIGS. 23 and 28. The differences in levels of protein expression were significant between C57BL and all lupus mice (P<0.01); while RDA-DNASE activity was slightly greater in the 89T strains than in the 891 strains.

FIG. 28. Graphs depicting D1L3 levels and DNASE activity in murine samples. The right column set in each display shows the mean D1L3 levels by immunoblot of serum and splenocyte/macrophage cell lysates for different mice strains and models. All graphs display from left to right: C57BL, BXSB, NZB/W F1, and MRL results. No anti-D1L3 immunoblot was performed for urine. The left column sets documents DNASE RDA activity measured in serum, urine, and splenoctye and macrophage-conditioned media. Mean values and standard deviations for all observations in FIG. 23. The results demonstrare that despite the D1L3 protein induction in splenocytes and macrophages is ineffectual in raising RDA-nuclease levels.

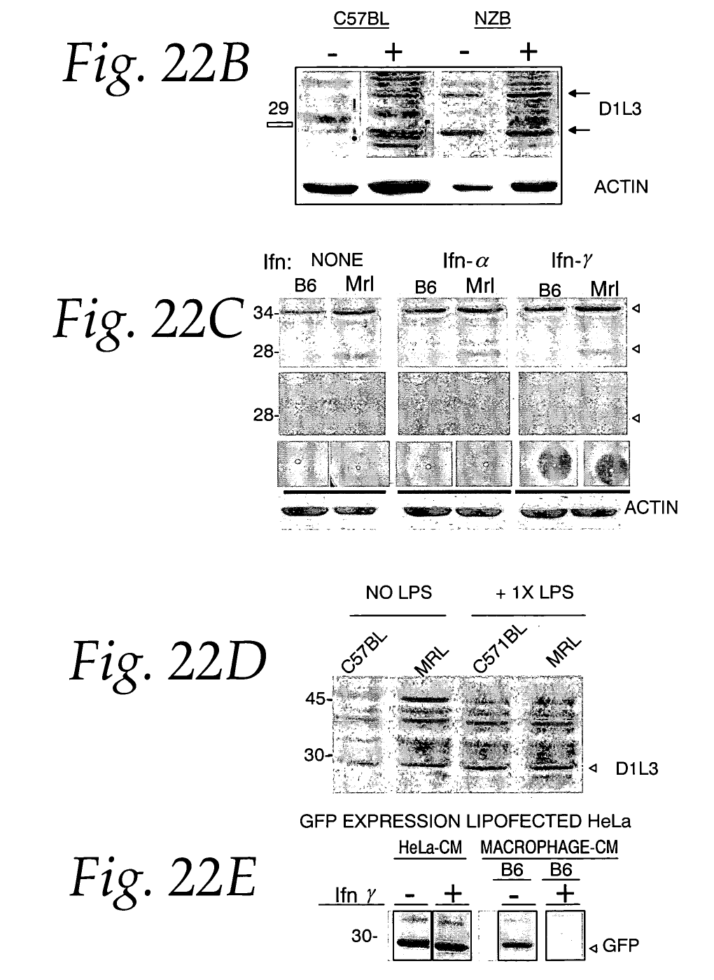

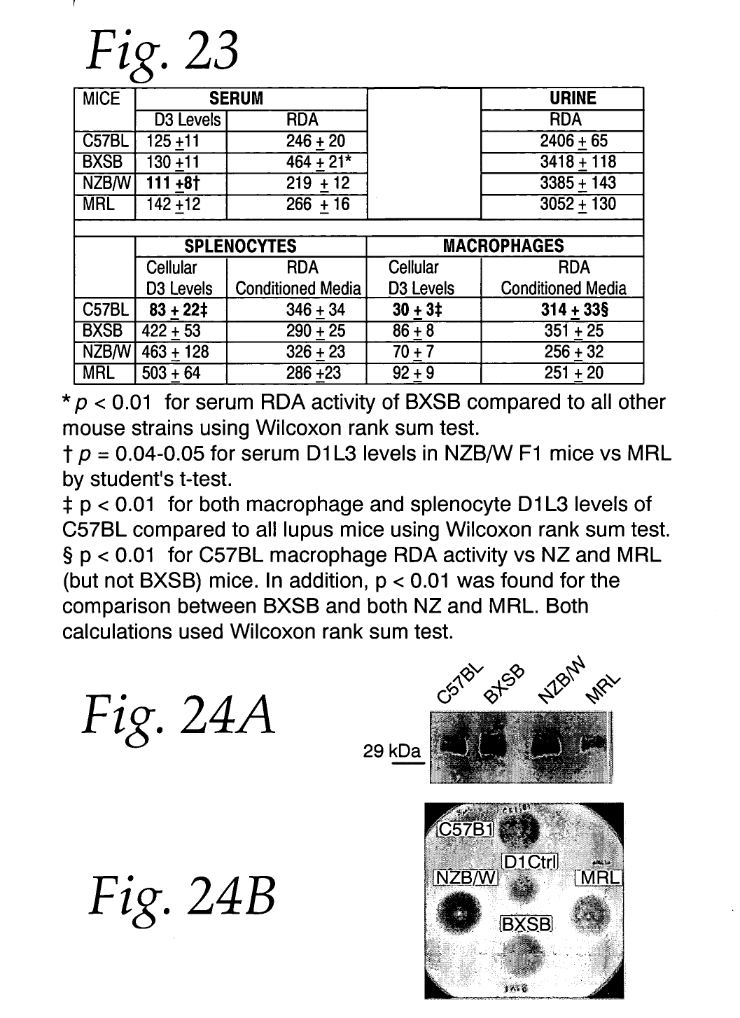

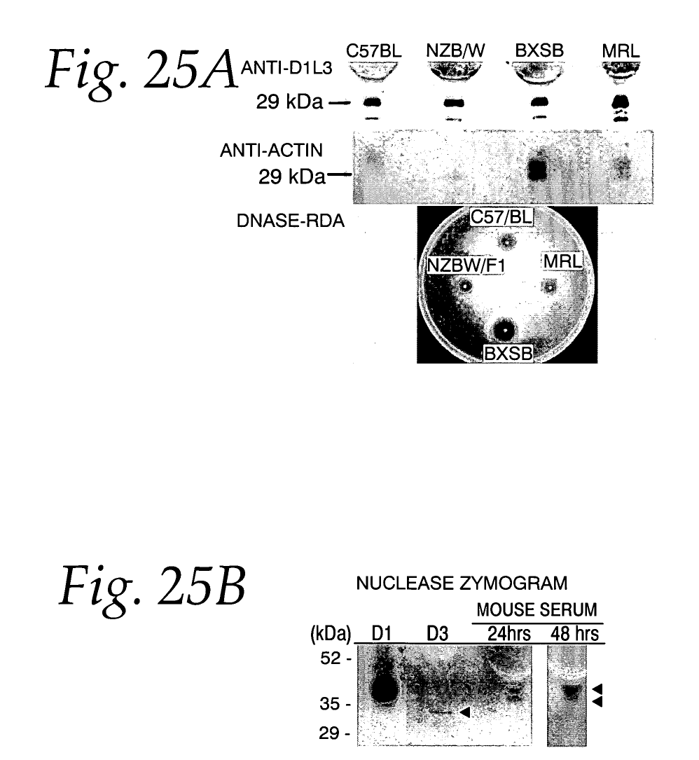

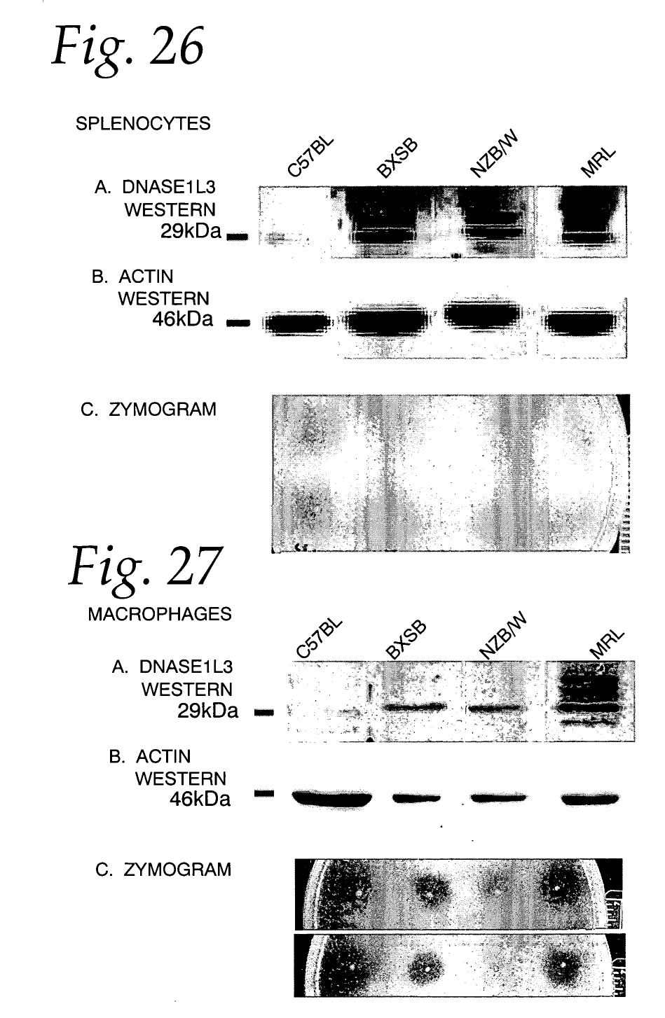

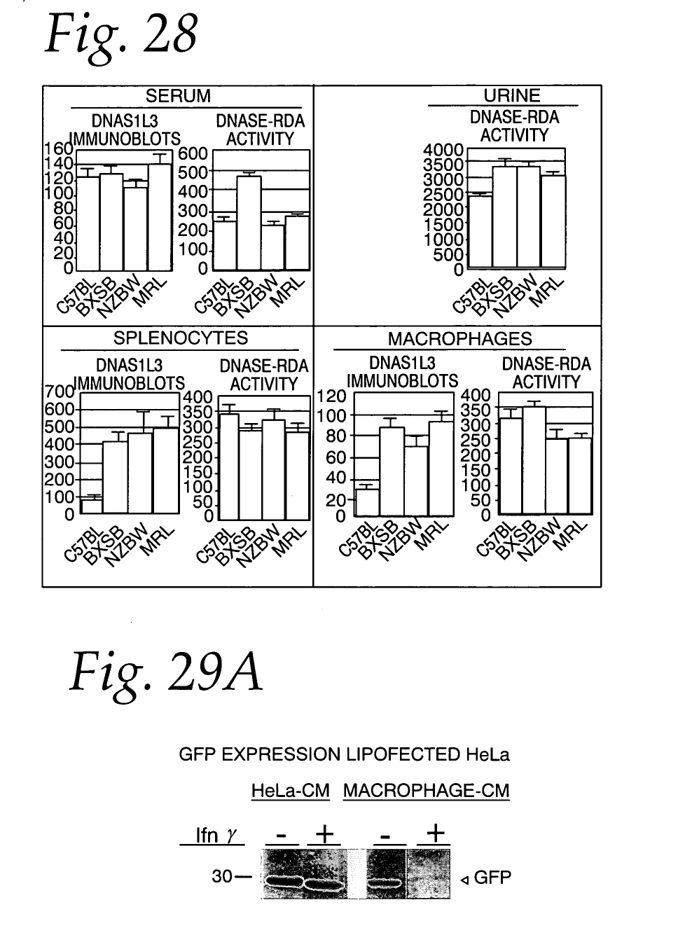

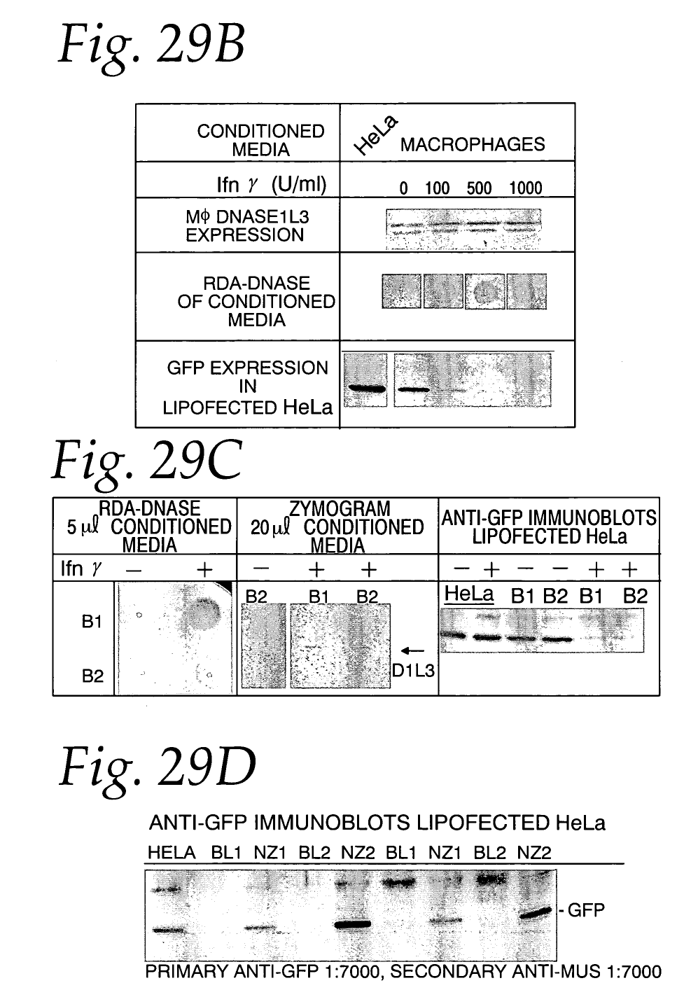

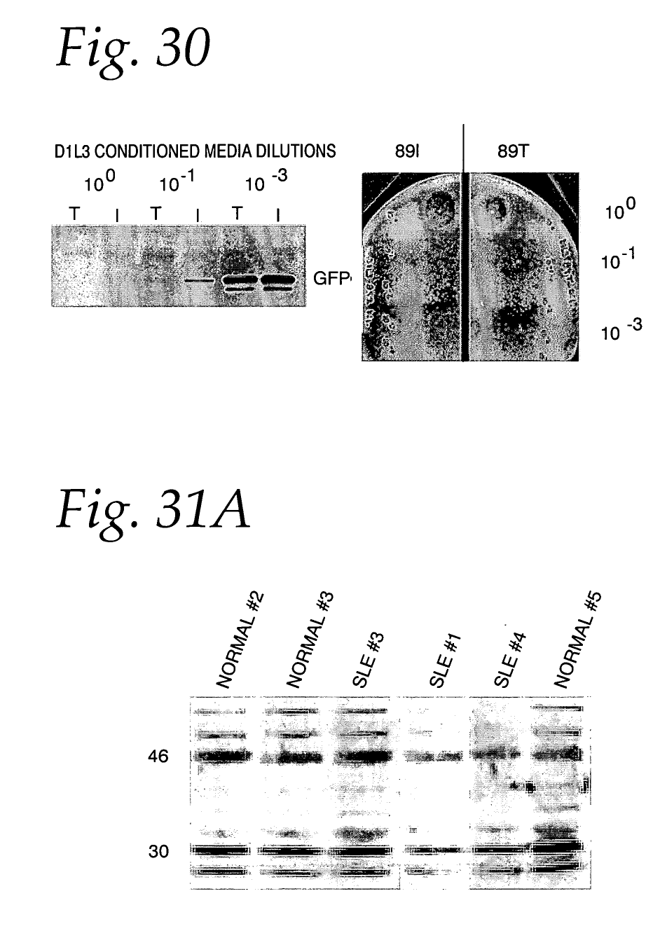

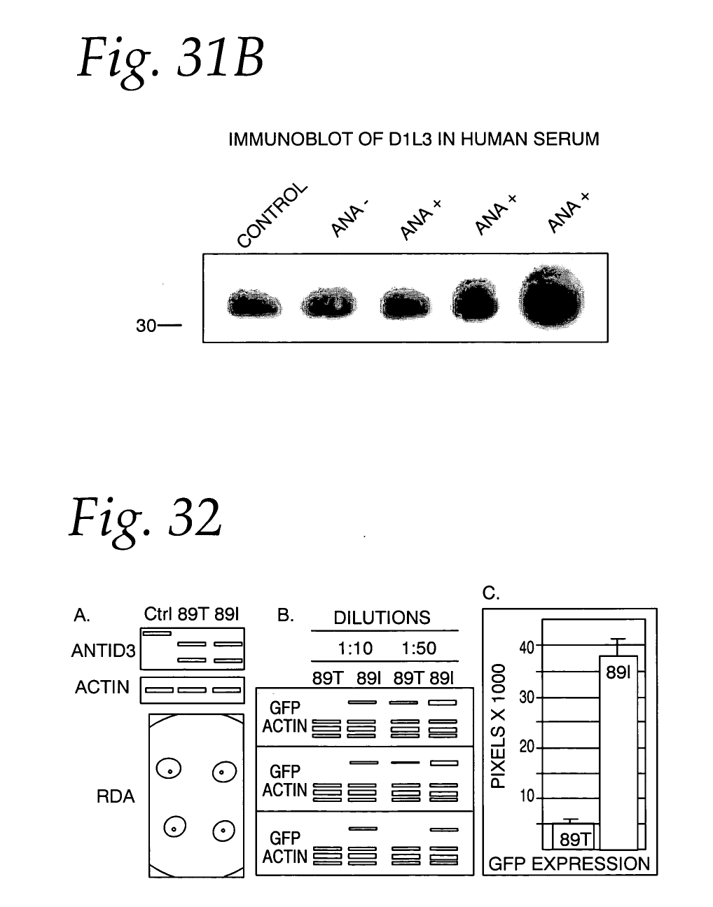

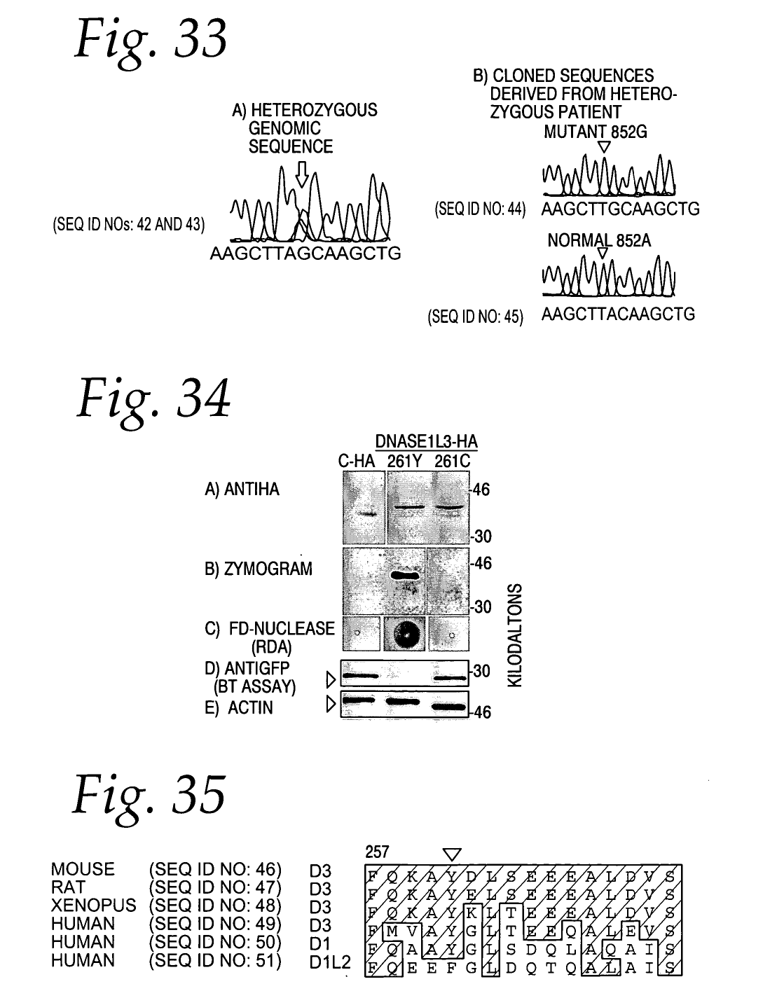

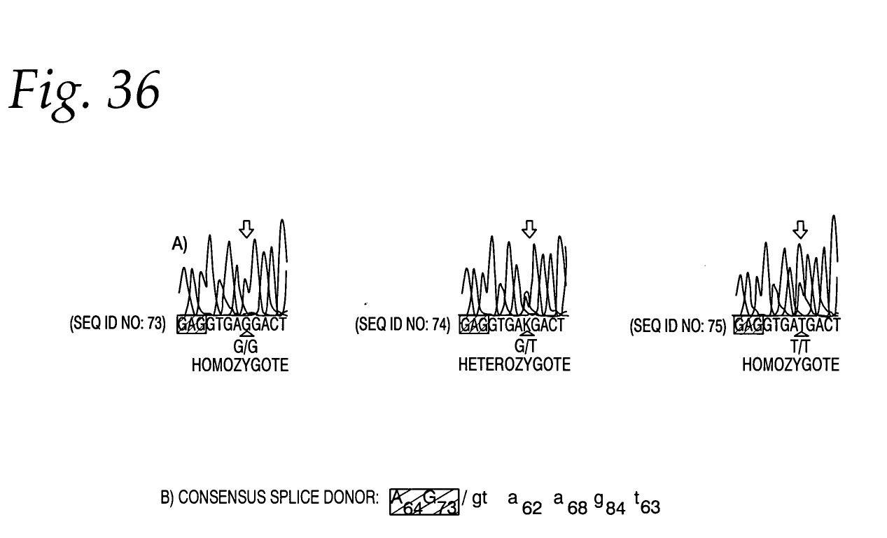

FIG. 29. Media conditioned by Ifn-γ treated macrophages from C57BI, but not NZB/W F1 mice is able to confer an in vitro barrier to liposomal gene transduction (BT). Panel A: Media conditioned by interferon-g (Ifn-γ) treated bone marrow-derived macrophages from C57 BL confers a barrier to liposomal transfection to HeLa. These results utilize the C57BL cells and media analyzed for FIG. 22C. Panel B: As interferon dose increased, the ability by C57BL macrophages to confer a barrier to transfection increased. In the absence of Ifn-γ, macrophages confer a weaker barrier. Panel C: Interferon gamma induces both DNASE activity by RDA and a DNASE band compatible with DNASE1L3 at 28 kD, plus induces a BT activity. Samples were from macrophages from two C57BL mice (B1 and B2). In the left register is pre- and post interferon RDAs, in the middle register, pre and post interferon, and in the right register are the BT assays (GFP immunoblots of tranfected HeLa cells) on HeLa cells, including HeLa cells treated directly with interferon gamma for 48 hours. Media conditioned for 48 hours. Panel D: BT activity in macrophage-conditioned media from NZB/W F1 (NZ) or C57BL (BL). Two mice from each class were sacrificed, and bone marrow derived cells were segregated into two wells each. Media was conditioned for 48 hours in the absence of GM-CSF, and then overlaid over HeLa cells. Wild-type macrophages are able to secrete or condition media with a barrier to liposomal transfection, while SLE mice are not. Interferon-gamma did not alter the results.

FIG. 30. D3-media confers an in vitro barrier to adenoviral gene transduction (BT). This figure provides anti-GFP immunoblots at 1:5000 (Clontech) of Ad.V.GFP infected (MO 100:1) of 100,000 HeLa cells. Infection of HeLa cells with Ad5.CMV-GFP (a 1st generation (DE1/DE3) adenovirus serotype 5 expressing GFP under the control of CMV-IE promoter (Qbiogene)) was performed in the presence of D1, D3, D1+D3CT, or control conditioned media. Infected cells were lysed at 72 hours and tested for GFP expression (n=3 expts). The immunoblots (n=2) demonstrate that D3- and D1+D3CT conditioned media prevents GFP expression, presumably by blocking infection. Thus DNASE1L3 or DNASE enzymes linked to the D3CT diminish adenoviral gene transfer. The increased expression of GFP in the D1 lane remains to be explained. Actin levels were generally equivalent in infected cells.

FIG. 31. 89I D1L3 differs from 89T by at least 10 fold in BT activity. When dilutions are made of D1L3-containing media, the ten-fold dilution conditioned media containing the wild-type 89T enzyme shows no GFP transfection efficiency, while the 89I shows transfection. Ample transfection is seen with a 1000-fold dilution of either, non with undiluted samples. RDA nuclease levels are show alongside. Results suggest a possible 10-fold difference in BT activity.

FIG. 32. Anti-human D1L3-peptide immunoblot of human peripheral leukocytes. Panel A: Protein lysates were prepared from histopaque purified peripheral mononuclear cells from human blood, and run on a 14% SDS-PAGE and immunoblotted with anti-human D1L3 peptide antisera, and secondary anti-Rabbit. Both cleaved and uncleaved bands are seen. Panel B: Immunoblot of D1L3 in human serum.

FIG. 33. Comparison of 89I and 89T BT activity. Compared by BT assay, the 89I enzyme is significantly defective. Normalized to immunoblotted D3 protein levels in pcDNA3.1-D3 transfected cells, 89I had a ^{˜}8 fold defect in BT activity (n=6, p-value =0.03 T-test). Panel A: FD-nuclease activity in the conditioned media was similar in 89I and 89T, because of a nearly 2-fold higher level of 89I in transfected cell lysates. Panel B: a ten-fold dilution of 89T nearly completely blocks GFP transduction, while liposomal gene transfection is possible in similarly diluted 89I-containing media. Panel C: Relative levels of GFP expression. Undiluted conditioned media in these experiments, which achieves higher levels of FD-nuclease activity than seen in Ifn-γ induced macrophage-conditioned media, conferred saturated BT activity with both 89I and 89T. These results establish that a significant primary defect in D3-BT activity is present in both the NZ and MRL strains of mice, and this finding in independent SLE models suggests a role in their susceptibility to the disease.

FIG. 34. Y261C human sequence traces. Sequence traces of Y261 C mutation and demonstration of heterozygozity. a) Sequence trace of the original heterozygous genomic amplicon. Similar heterozygozity was observed on reverse strand. b) Sample was re-amplified from original stock, and cloned into TA vector. Six individual clones were isolated and sequenced; two of the clones depicting either mutant (n=4) or normal (n=2) sequence are shown.

FIG. 35. Y261 C mutation causes complete loss of D3 function A human DNASE1L3 clone (derived from EST 82269, (31), fused in frame to hemagglutinnin tag (HA) was used for this analysis. Mutagenesis of the clone was performed using QuikChange kit (Stratagene) and the sequence-verified pcDNA-3.1 derived HA-tagged clone was expressed in HeLa. a) Anti-HA blots with 12AC5 (1:200) showed no difference in expression of the DNASE clones (data not shown). C-HA control lane was transfected with parental vector. b) The mutant 261 C showed no activity by zymogram, and only faint, if any, activity by RDA (c), and BT assay (anti-GFP) showed an absence of barrier activity (d). e)Actin lane shows equivalent protein loads for BT assay.

FIG. 36. Alignment focused on DNASE1L3 residues at 261 showing high degree of conservation

FIG. 37. IVS6+5 homozygotes and heterozygotes. a) Sequence traces of (left to right): wild-type GG homozygote, GT heterozygote, and TT homozygote. Arrows illustrate polymorphic IVS6+5 nucleotide. The 3′ end of exon 6 is shaded gray. b) Array represents consensus splice donor region. Numbers below are the percent of verterbrate introns with a given residue at that position; thus, the +5 nucleotide is G in 85% of exon junctions.

### DETAILED DESCRIPTION

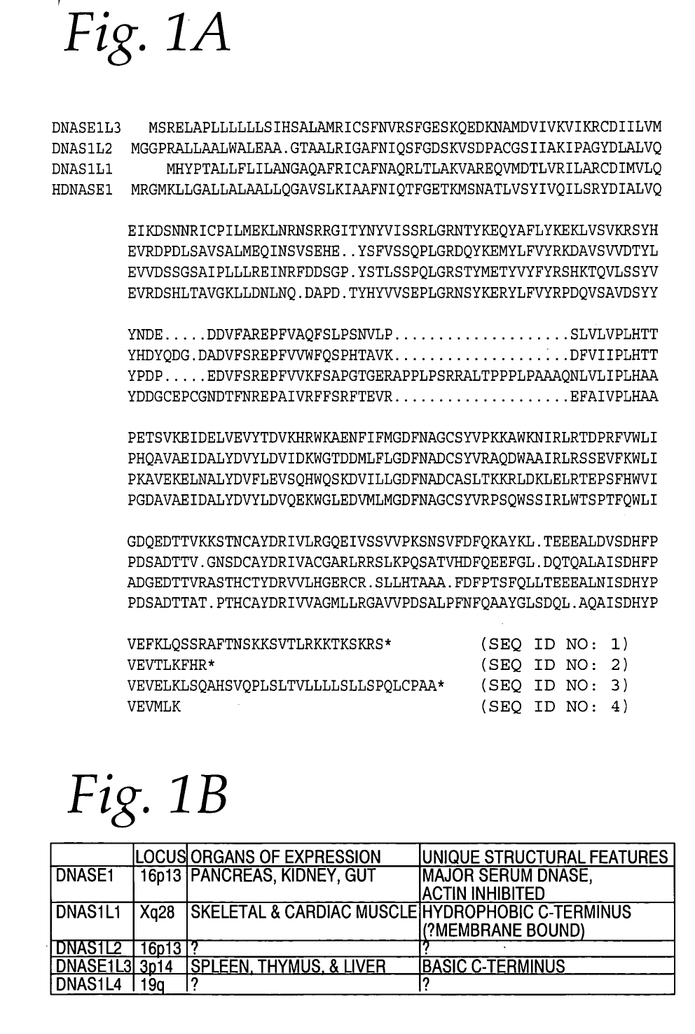

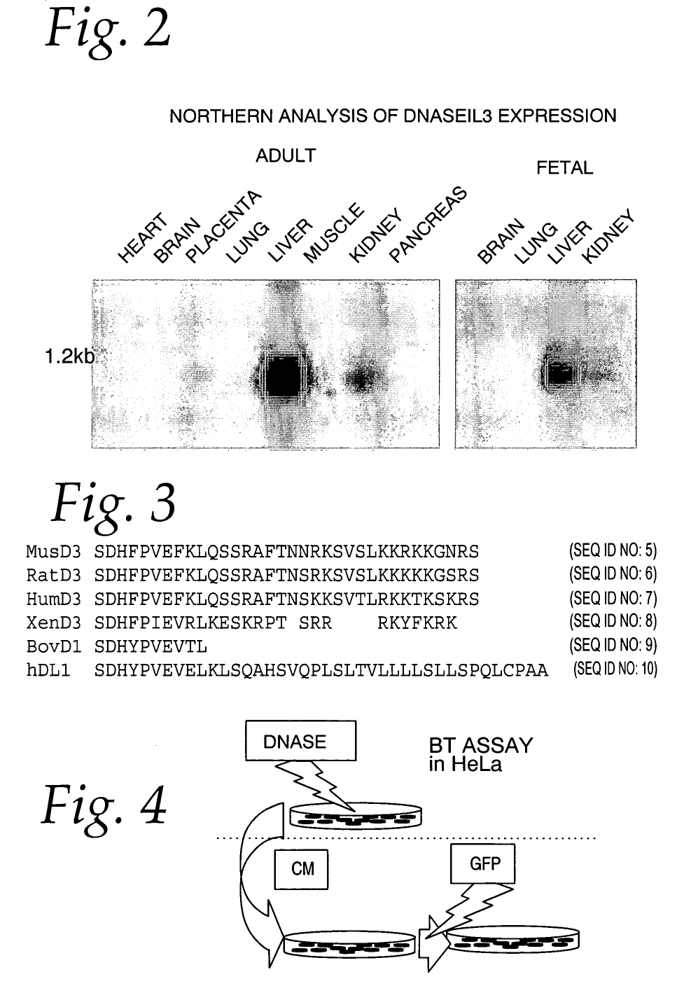

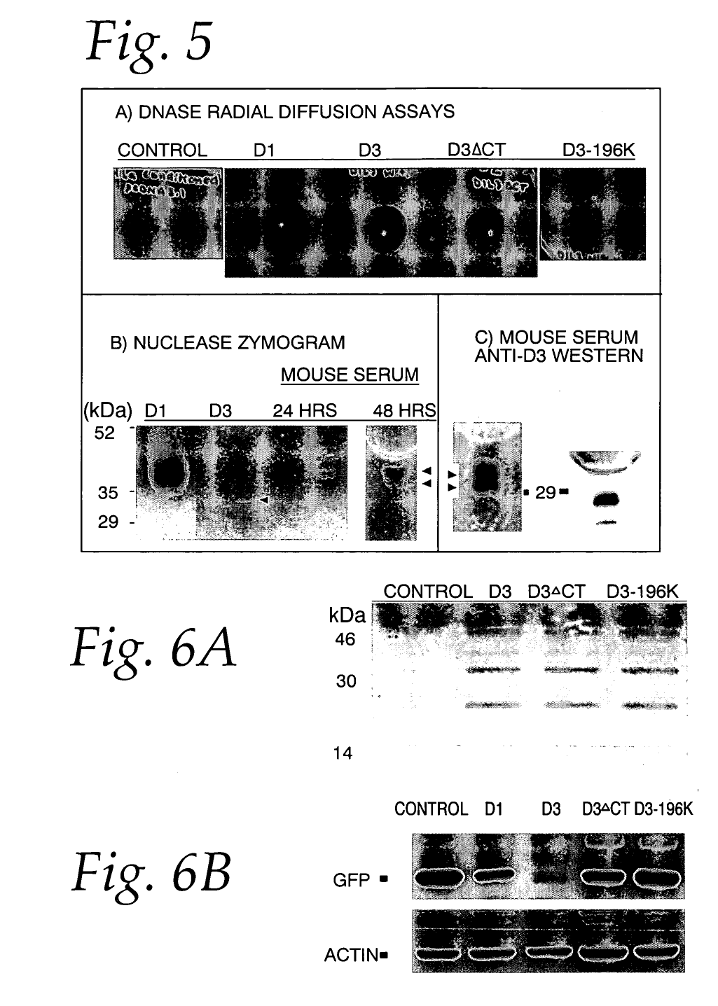

DNASE-1-like 3 (DNASE1L3 or D1L3). The human DNase gene family consists of 4 homologous, yet distinct loci, with different tissue expression patterns (Shiokawa et al., (2001) Biochemistry 40, 143-52; Rodriguez et al., (1997) Genomics 42, 507-13) (FIGS. 1A, B). D1L3 expression by northern was prominent in liver (FIG. 2). The protein was immunodetected in liver, spleen, and thymus, where it is mostly present in macrophage-derived (adherent) populations (FIG. 22). By western, D1L3 expression is found in spleen, liver, and thymus. In addition, the enzyme is found by western blot mainly in adherent cell populations from spleen (presumably macrophages) (FIG. 22A and Baron, 1 998 #4) and serum (FIG. 25). The enzyme may also be produced by other thymocytes and non-adherent splenocytes. The enzyme is secreted and concurrent zymograms of cell lysates and conditioned media of transfected cells confirm D1L3's signal peptide is cleaved, as are the other DNASEs.

The full biologic functions of D1L3 are unclear. Apoptotic endonucleic “laddering” of lymphocytes to intracellular DNAS1 L3 (Shiokawa et al., (2001) Biochemistry 40, 143-52) has been attributed to this enzyme. In cultured macrophages, the enzyme and the activity in conditioned media are inducible by Ifn-γ, LPS, and phorbol myristate acetate, all agents that promote monocyte differentiation and activation. Lipofection-associated DNA does show some laddering with D1L3 exposure (FIG. 16). If D1L3 is crucial for oligosomal degradation of apoptotic DNA, the absence of this macrophage-secreted nuclease from cultures of most cultured cell lines may explain why apoptotic DNA ladders are less consistently observed there versus primary cell cultures. These studies suggest this apoptotic hallmark, thought to reflect only intrinsic enzyme activation, could be acquired via D3-entry into cells from surrounding milieu. D1L3 is easily detectable in serum by immunoblot; therefore, the BT effect is likely distributed throughout tissues. Since the nuclease levels by RDA and the levels of immunodetectable D1L3 in serum are similar or lower to those generated in the conditioned media of the described experiments; an in vitro D3-BT effect is likely to be present in serum in vivo.

The human D1L3 gene is located on human chromosome 3p14-3p21 and in the syntenic location on mouse 14. Herein, D1L3 may refer to native D1L3, or natural, synthetic or recombinant variants thereof that retain the D1L3 activity. Unless otherwise specified, D1L3 herein refers to D1L3 that is found in any source, including murine, bovine, ovine, porcine or human. Unlike D1, native serum D1L3 has a basic C-terminal extension and does not have glycosylation sites. D1L3 is described in more detail in Genomics 42:507-513 (1997), which is hereby incorporated by reference. Murine D1L3 extends 21 residues (289-310) further than the aligned D1 enzyme (nearly half these amino acids are basic (arginine or lysine). FIG. 3 aligns murine D1 and D1L3 enzymes from mouse, human and xenopus, respectively, starting at the highly conserved motif, SDH, and shows the conserved basic character of this stretch. Although the core D1L3 is highly homologous to D1 and DNase 1-like 1 (D1L1), D1L3 activity differs from D1 activity in that D1L3 effectively blocks liposomal transfection (lipofection), while D1 does not. This novel DNase 1 L3 activity is an in vitro BT, and in vivo likely forms part of a cellular shield to the nuclear acquisition of exogenous DNA.

Studies of in vivo Effects of D1L3:

D1L3 Is a Barrier to Liposomal Gene Transfection: Definition of BT Activity. Liposomal transfection is less efficient in vivo than in vitro (Crystal, (1995) Science 270, 404-10). While DNase1 (D1) has been implicated as a prime candidate for imparting barrier to transfection, this nuclease has little in vitro activity on liposome-complexed DNA. More significantly, D1-conditioned media has little effect on in vitro transfection efficiency. On the other hand, media conditioned with D1L3 provides a potent barrier to in vitro liposomal transfection and may be the in vivo basis for barrier to transfection (BT) in wild-type organisms.

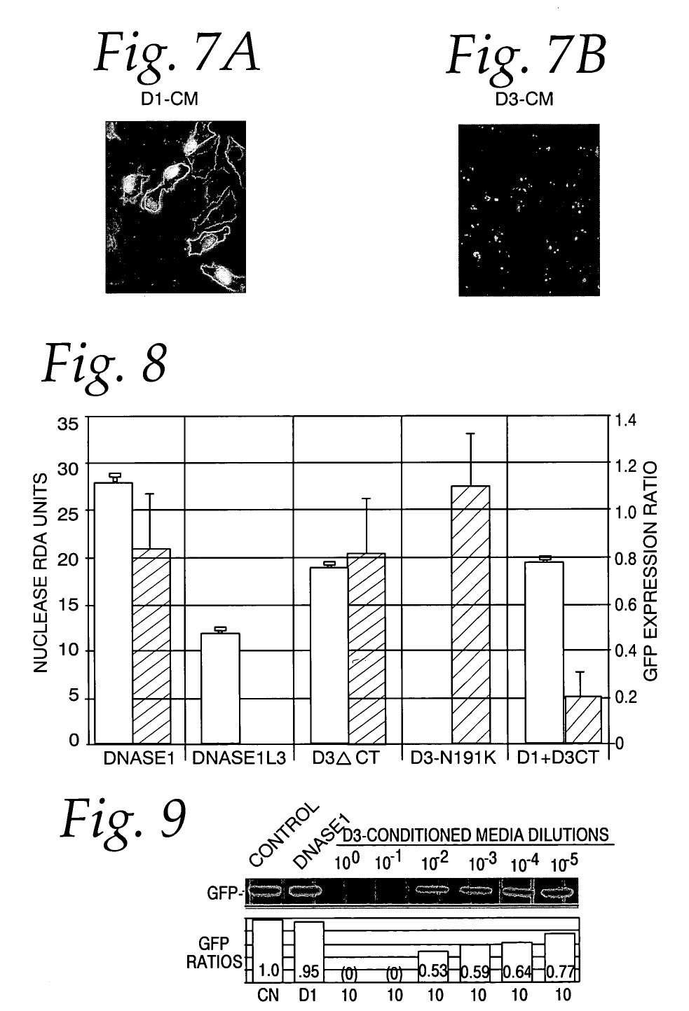

The experiments summarized in the figures reveal that D1L3-conditioned media (CM) generally ablated expression of GFP in the lipofection studies, while GFP expression did not differ greatly between cells incubated in either control of D1 conditioned media. In all, D1 conditioned media had more nuclease activity against free DNA by RDA than D1L3 conditioned media. By densitometry of RDAs, D1 media contained a mean of ^{˜}2.7 fold (SD=0.8; n=24 assays; p<0.001) more activity by RDA than D1L3. In FIG. 8, the values were obtained from scanned tiff figures used to calculate pixel “volumes” of RDA activity (area×pixel darkness). D1 and D1L3 conditioned media were assayed in parallel on the same agarose dish. Inter-experimental incubation conditions were nearly identical (24 hours, 37 degrees, same concentration DNA and agarose). D1 media contained a mean of 28.2 Units of activity (SD=0.7) versus only 12.0 (SD=0.2) for D1L3; n=24 assays; p<0.001). The mean ratio of GFP expression in D1-exposed cells versus control was 0.8+0.2 (n=20 assays). Actin immunoblots did not differ among samples, showing equal protein loading (FIG. 6). No GFP expression was seen in D1L3 exposed cells. Fluorescent microscopy at 24 hours confirmed the absence of GFP fluorescence in cells incubated in D1L3 conditioned media, while abundant GFP can be visualized in D1 conditioned media exposed cells (FIG. 7). Zymograms of the respective CM demonstrate the expected bands of activity for both the DNases, with glycosylated D1 showing a larger, more intense, and diffuse band than D1L3.

In the main assay, an eukaryotic marker such as green fluorescent protein (GFP) expression plasmid complexed with a lipid reagent is transfected into HeLa cells incubated in control media, D1L3-conditioned media, or D1-conditioned media (FIG. 4). The assay is not limited to the use of GFP as a marker, but could use any of many expressed biomarkers encoded by nucleotides that can be assayed either immunochemistry, fluorescent detection, or enzymatic detectection, and these include but are not circumscribed to luciferase, chloramphenicol acetyl-transferase, antibiotic or chemical resistance genes, Beta-galactosidase, FLAG-, myc-hemaglutinnin-, or other epitope tagged molecules, as well as other markers of gene or nucleotide transduction. The endpoint of the assay is the detection of a synthesized protein, but could easily have been the detection of a transcribed gene product, since the assay ultimately measures the ability of D1L3 added the media/milieu overlaying the cells to block the ultimate association of an expressible plasmid with the transcriptional machinery.

The ability of an agent to overcome the BT effect reflects the ability of the agent to overcome the barrier to the passage of the transduced agent from outside of the cell into an intact and active transcriptional site, presumably nuclear. The exact site of action of D1L3 (or D1+D3CT) is unknown; the data suggests that glycosylation leads to the targeting of a less effective site, likely lysosomal. The experiments also indicate interaction with cells appears to be required. The present invention provides a gene therapy composition comprising a recombinant gene expressing D1L3-like barrier to transfection and a lipofection reagent. Media conditioned with such enzymes will prevent liposomal transfection, and can be used to test, which transfection reagents overcome this barrier. The enzyme can be provided to the media by either expression of a gene containing the full-length sequence of D1L3 such that the enzyme can be secreted in eukaryotic cells. In addition, the enzyme can be synthesized and purified from other systems such as bacteria, yeast, tissues, and cell culture, and isolated in a glycerol-stabilized format. Another endpoint that could be used in assays to overcome the BT effect could also be the ability to provide distinctive capacity to a cell line via transient or permanent modification of the genome, for example ability to survive or permit selection in a specific milieu. The goal of such assays would be the derivation of more effective liposomal transfection reagents.

Deoxyribonuclease 1-like 3 (D1L3) hydrolyzes lipid-complexed DNA and decreases transfection efficiency in liposomal transfection (lipofection) systems. As such, D1L3 provides a better test for the efficiency of lipid/liposomal based gene therapy than current standards using deoxyribonuclease 1 (D1). Moreover, blocking D1L3 activity enhances liposomal transfection for gene therapy, while increasing D1L3 activity may enhance destruction of pathogenic DNA, whether viral, bacterial or endogenous, thereby providing treatment for lupus, or viral and oncogenic diseases. In addition, D1L3 provides a better test for the efficiency of lipid/liposomal based gene therapy than current standards using deoxyribonuclease 1 (D1).

Potent BT Activity Can Be Conferred by Conditioned Media. Similar results are obtained regardless whether the secondary GFP transfection was performed on the cells expressing D1L3 and D1 (n=2). Thus, the BT effects occurs whether D1L3 is expressed by cells or found in the media, and D1 lacks a BT effect in both cases. In addition, GFP transduction is also blocked, when naive cells are incubated in D1L3 media for six hours, rinsed twice with PBS, and then subjected to transfection in control media. This suggests the D1L3 does not need to be directly in high concentration in the media to be of effect. A complete BT effect was still evident when D1L3 conditioned media was diluted 100 fold with control media or 10-fold with N196K conditioned media (FIG. 10). At these dilutions, the nuclease activity by RDA is not clearly distinguishable from background (data not shown). The levels of nuclease activity by RDA in undiluted D1L3 conditioned media are similar to those observed in equivalent volumes of C57BL murine sera; however, it must be acknowledged that actin inhibition experiments and zymogram analysis strongly suggest DNASE I comprises substantial portion of serum activity (data not shown). A substantial BT effect is still at 100-fold dilution (FIG. 9).

The novel observed BT-activity also does not require intrinsic expression of D1L3. This suggests circulation of this macrophage-secreted enzyme in the serum can distribute this protective effect throughout tissues. Thus, these observations of an in vitro BT effect are likely to be true also in vivo, where D1L3 shares the serum compartment with D1.

Therefore, blocking D1L3 activity may increase effectiveness of certain gene therapies. Peptide or monoclonal blocking agents against D1L3 may protect lipid/liposome based transfection systems. This is useful, particularly near areas of circulation, because non-viral gene transduction is believed to be less toxic than viral transduction.

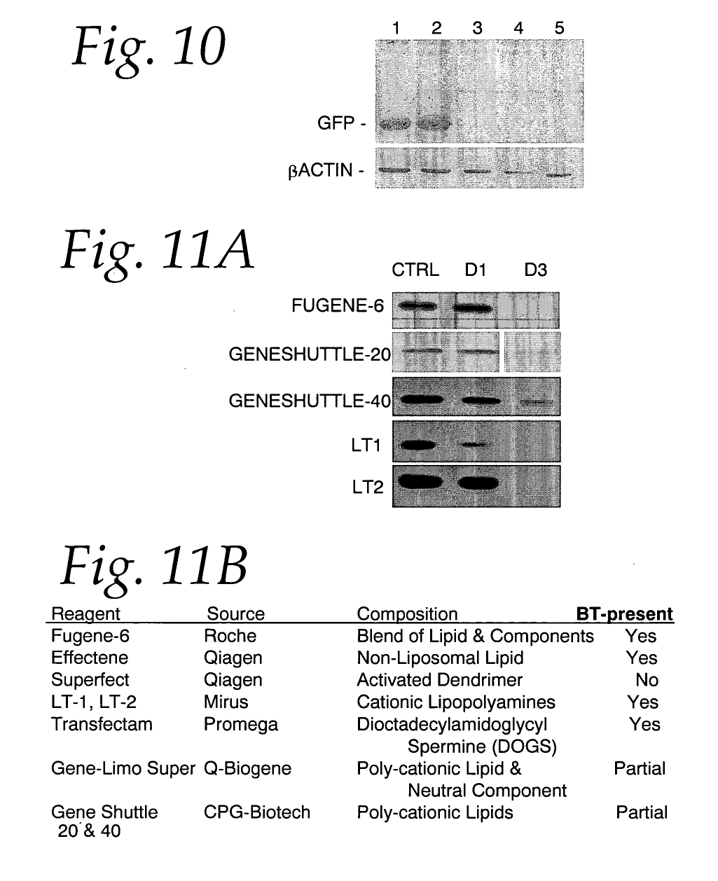

BT Activity Occurs with Various Liposomal Transfection Reagents. Most of the experiments were performed using FuGENE™-6 (Roche Diagnostics Corp.; Indianapolis, Ind.). The studies show that D1 has no effect on the transfection efficiency of liposomal transfection systems such as FuGene™-6, while D1L3 effectively blocks transfection efficiency in FuGene™-6. However, using accompanying protocols, a variety of transfection reagents (FIG. 11B, n≧2 for each reagent) were tested. Examples include the non-liposomal lipid-based Effectene (Qiagen); the cationic lipopolyamines-based LT-1 and LT-2 (Mirus); the dioctadecylamidoglycyl spermine (DOGS)-based Transfectam (Promega); the polycationic lipid/lipid mixture of Gene-Limo Super (Q-Biogene); and the polycationic lipid-based GeneShuttle 20 and 40 (CPG-Biotech). A BT effect was observed with all polycationic lipid reagents. With GeneShuttle and GeneLimo reagents (n=2 experiments), the effect of D1L3 conditioned media was partial; however, the pattern was still similar (D1L3 GFP expression <D1 or control). Only the non-liposomal dendrimer-based Superfect reagent (Stratagene) did not demonstrate a BT effect. The DNA associated with this reagent may not be susceptible to the BT nuclease effect because either the DNA enters cells through an a typical non-endosomal pathway or the branching dendrimer, which has rather different composition than lipid-based reagents, excludes the access of D1L3 to the enzyme (Kukowska-Latallo et al., (1996) Proc. Natl. Acad. Sci. USA 93, 4897-902). These experiments were not meant as a definitive assessment of the susceptibility of various reagents to the D1L3-BT effect, but to demonstrate that this novel enzyme activity occurred with all polycationic liposomal reagents tested.

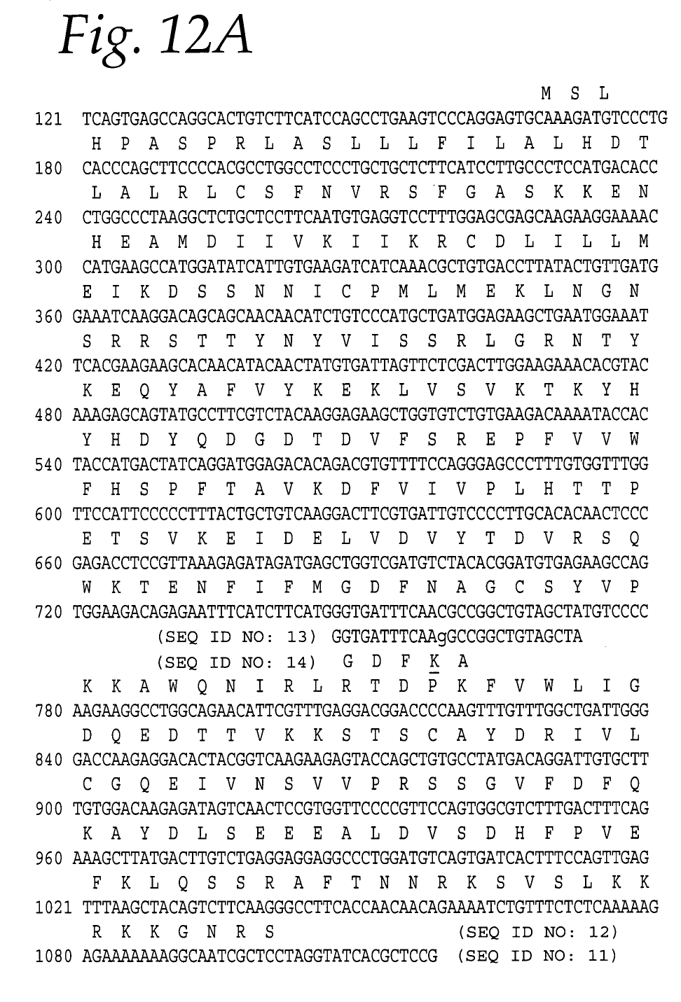

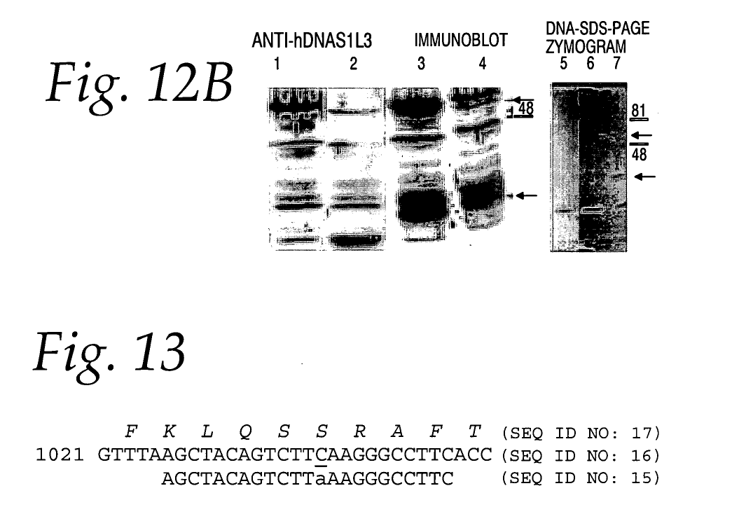

Requirement of Nuclease Activity for BT Activity. To determine if nuclease activity, as expected, is required for BT activity, the highly conserved asparagine in D1L3 residue 196 was changed to lysine (N196K). This residue is identically represented in a highly conserved AA motif (GDFNA) in all mammalian DNases. In human D1L3 the analogous mutation (N191K) shows no detectable nuclease activity by zymogram (FIG. 12B). The role of this motif acts in DNA nucleolysis is unknown; the motif was not identified in crystallography experiments as participating in DNA-enzyme contact, nor has it been studied in mutagenesis experiments. However, the near-invariant identity of GDFNA in nearly all DNASEs predicted an important role in DNASE function, and it was therefore mutated to create an inactive or non-functional enzyme.

As expected, the N196K-D3 has no nuclease activity above background by either RDAs of conditioned media or zymograms of cell lysates, and is incapable of providing a barrier to transfection. N196K and wild-type protein size and levels are equal in cell lysates (FIG. 6). There is no a priori reason to expect altered secretion of the mutant enzyme. It is likely that all missense mutations that abolish or alter the nuclease activity of the enzyme by RDA, would similarly affect BT activity. These include mutations that alter residues that bind calcium, catalyze nucleolysis, and DNA-binding.

Thus BT activity appears to require an intact deoxyribonuclease, and is not the result, for example, of other qualities of the protein. This result also suggests that compounds that block D1L3 DNASE activity, for example, ions (zinc), chelating agents (EDTA), or aurintricarboxylic acid (Shiokawa et al., (2001) Biochemistry 40, 143-52; Shiokawa et al., (1997) Arch. Biochem. Biophys. 346, 15-20; Shiokawa et al., (1997) Biochem. J. 326, 675-81) or inhibitory peptides or proteins, could be used to inhibit the BT activity. Unlike DNASE i, no specific protein inhibitor of D1L3 is known.

Requirement of the D1L3 C-terminus for BT Activity. While homologous to D1 in its core nuclease sequence, D1L3 has a distinctive highly basic C-terminal stretch, which resembles both the basic nuclear localization signal like those from SV40 (Dingwall et al., (1991) Trends Biochem. Sci. 16, 478-81) as well as polylysine stretches often found in polycationic liposomal reagents. To test the requirement of the D1L3 C-terminus in the BT effect, a point mutation was created in the full-length clone, such that a stop codon is substituted for the codon for Ser-289. The amino acids 289 to 310 (289-end) of the murine D1L3 are hence called the D1L3 C-terminus or D1L3-CT or D3CT). Point mutagenesis was achieved using primer D3D289-310 and its reverse-complement, and confirmed the mutation by sequencing and restriction digest (see methods) (FIG. 13). The primer mutates base pair 1038 from a cytosine to adenine; thus substituting a TCA (Ser) with a TAA (stop). The mutagenized clone would encode D3DCT, a truncated enzyme, which retains the motifs common to all DNases and thus, as predicted, retains nuclease activity.

By RDA, D3 pCT conditioned media had slightly increased nuclease activity relative to D1L3 (FIGS. 6 and 8). GFP transfection efficiency, however, was unaffected in cells were exposed to D3 ρCT conditioned media. Thus, the truncated enzyme retains DNASE activity, but lacks BT activity. This strongly suggests that BT activity by D1L3 is not merely a function of its core nuclease domains, but requires the cis attachement of the D1L3 C-terminus.

D1 Fused to the D3CT Can Confer a Barrier to Liposomal Gene Transfection. The nuclease sequence required for BT activity is not specific to D1L3. The entire N-terminal sequence (1-289) of D1L3 can be replaced with D1, and the fusion protein still evidences BT activity. Other DNASEs, and perhaps nucleases, likely could also fulfill the role, provided that they can access and be active in the same extracellular compartment as D1L3. The experiments suggest a DNASE activity, regardless of the origin, needs to be linked to the D3-C-terminus, for BT activity. It is possible ribonucleases, exonucleases, or site-specific endonucleases could be targeted to lipofected DNA or infectious DNA/RNA by addition of a D3-C-terminus. While it has not been demonstrated, which enzymatic activities can be efficiently replaced, and what the functional characteristics these enzymes can have the principle that an enzyme linked to a D1L3 C-terminus will have some access to lipofected material, or material resembling liposomal material is hence suggested.

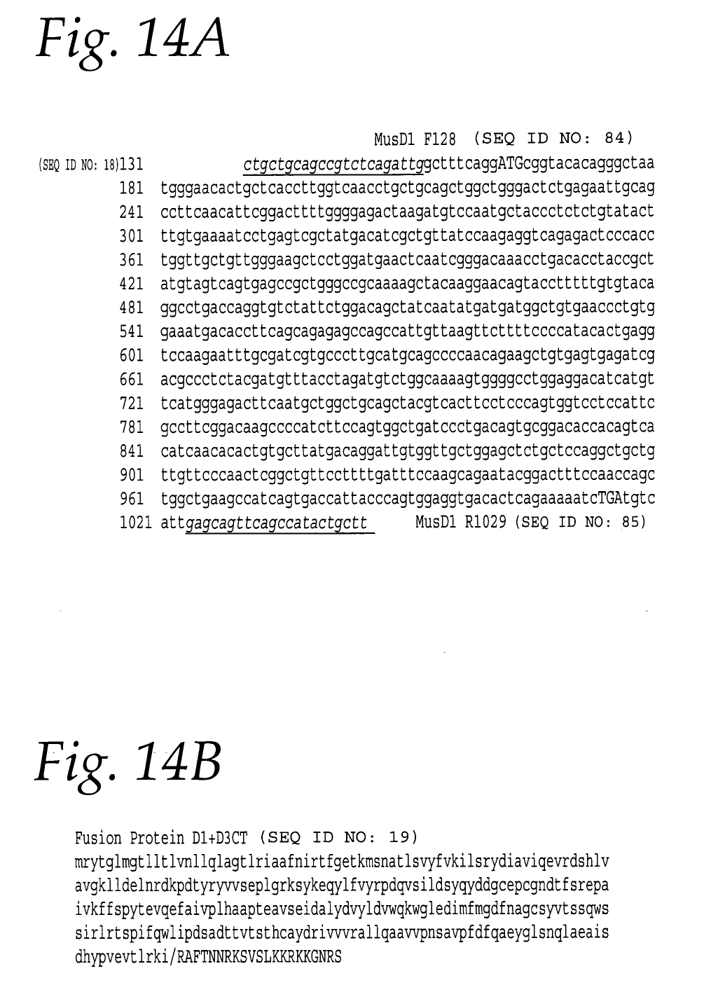

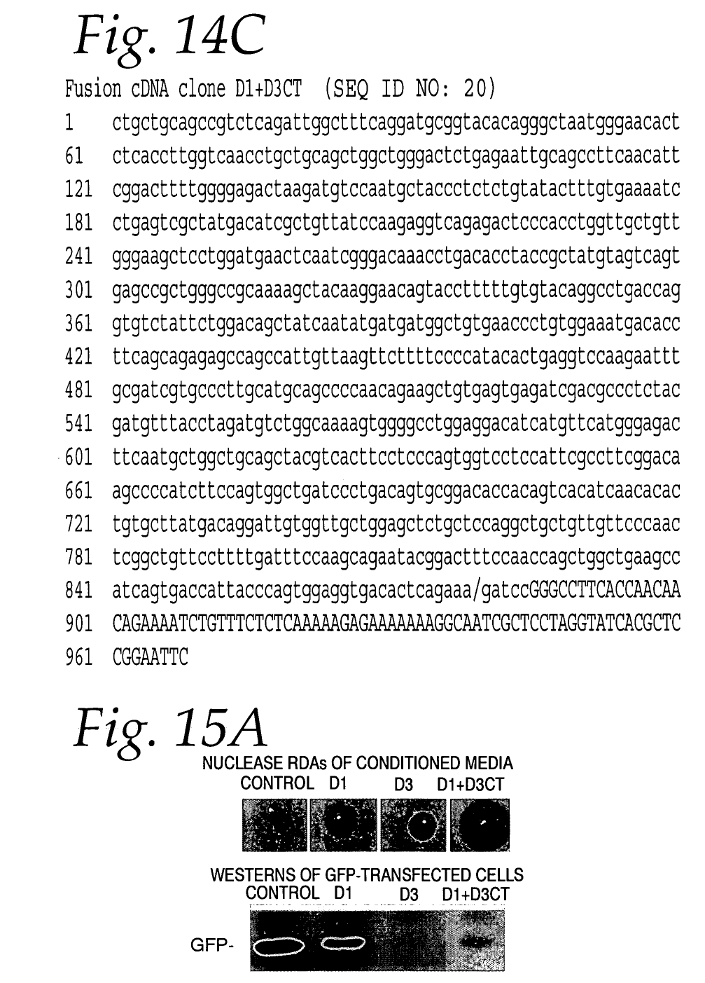

The present invention also demonstrates how a chimeric DNase (D1+D3CT) comprising DNase1 fused in frame to D1L3 C-terminus (D3CT) can mediate BT activity (FIG. 14). All murine cDNA sequences used for expression experiments were derived from C57BI. This experiment shows that no D1L3-specific sequences from AA 1-289 are essential to BT activity, other than a DNASE. That is, these results confirm that the core D1L3 nuclease residues upstream of the C-terminus are not essential for BT activity. For an enzyme to have BT activity, an extracellular DNase covalently linked to the basic C-terminus of D1L3 suffices.

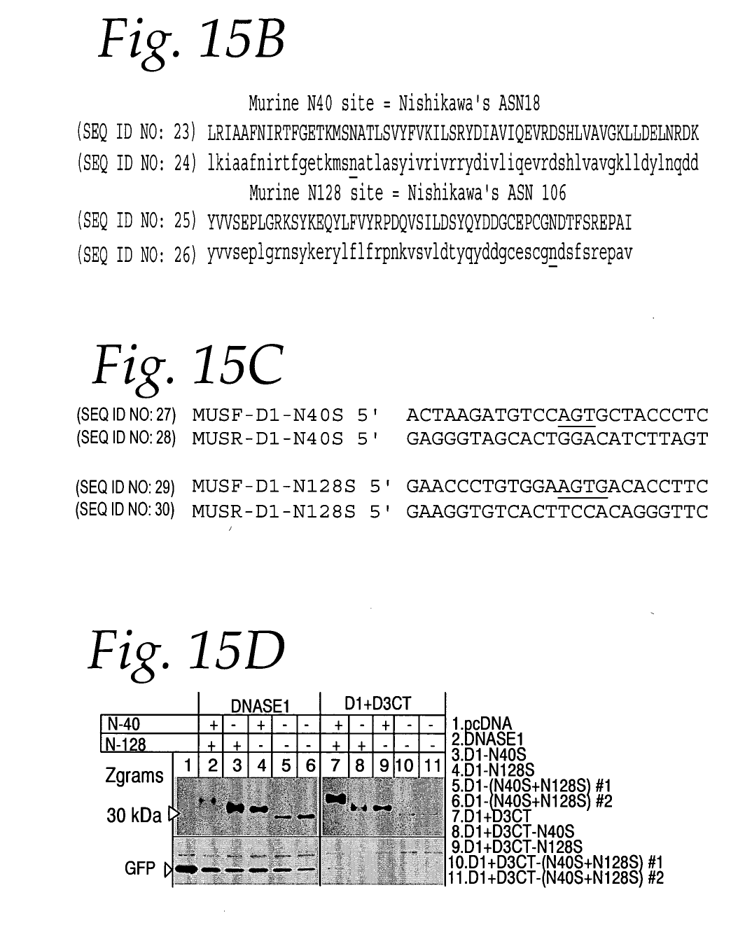

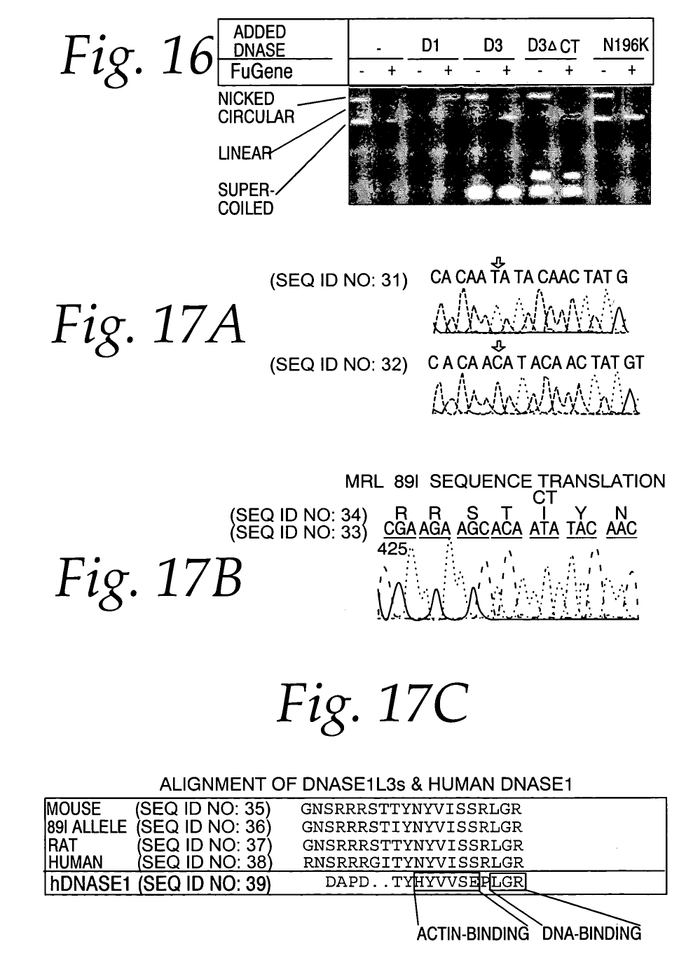

D1+D3CT was engineered by fusing N-terminal D1 (stop codon altered) in frame to D3CT. D1+D3CT conditioned media in fact had greater mean RDA activity than D1 (3.4±0.8 fold, n=4) D1+D3CT-media had greater mean RDA activity than D3 (19.6 Units±0.33 Units, n=4), but more importantly, the D1+D3CT shows significant BT activity, lacking in parental D1. D1+D3CT conditioned media treated cells show significantly less GFP expression than control cells (FIG. 15A, n=4 westerns) than cells exposed to control conditioned media. Since all experiments are performed by transfection of equivalent amounts of DNA, the larger areas of clearing on RDA and zymogram strongly suggest greater activity. It remains to be shown whether equivalent masses of expressed protein also show this activity.

The BT activity of D1+D3CT can be enhanced further by mutagenesis of known N-linked glycosylation sites on DNASE I. N-linked glycosylation sites are Asn-Xaa-Ser/Thr (or N-X-S/T where Xaa not proline). Studies of the glycosylation of D1 for the murine and bovine enzymes have been previously reported (Nishikawa et al., (2001) Biochem. J. 355, 245-8; Nishikawa et al., (1999) J. Biol. Chem. 274, 19309-15; Nishikawa et al., (1997) J. Biol. Chem. 272, 19408-12). The murine N40 and N128 sites correspond respectfully to the ASN-18 and ASN-106 sites described by Nishikawa for the cleaved bovine enzyme (FIGS. 15B,C). The N40 site is nearly always glycosylated, second is up to 70% glycosylated in vitro COS cell expression experiments (Nishikawa et al., (2001) Biochem. J. 355, 245-8). Point mutagenesis, conservatively substituting the asparagines with serines, was performed on DNASE1 (FIG. 15). The residues are found only in DNASE I and not conserved in other DNASE family members, hence not predicted to be essential for nuclease activity.

The experiment confirms both sites can be glycosylated, and sequential ablation of both sites creates a sharper smaller band of DNASE activity (−26 kDa). Not unexpectedly, as the chimeric D1+D3CT enzyme loses glycosylation motifs, and hence resembles D1L3 more, it has increased BT activity (FIG. 15D).

Glyscosylation with mannose residues likely diverts D1 to a lysosomal pathway (Cacia et al., (1998) Biochemistry 37, 15154-61), and suggests D1L3 BT activity operates along a different pathway to the nucleus. Lysosomal degradation by D1 and DNASE II may be the main fate of most of the endocytosed liposome-associated DNA; however, the D3CT guided pathway may protect the nucleus from DNA entering the cell either through non-lysosome targeted compartments (for example, caveolin- or clathrin associated pathways. Deletion of the glycosylation sites from D1+D3CT creates a chimeric enzyme that is now free from lysosomal targeting. Alternative reasons why deglycosylation enhances BT are that glycosylation may interfere D3CT effect by interacting with that domain or with other molecules at the site or access to D3-BT activity.

The observed phenomenon has implications for the planning and development of more efficient enzymes with BT activity. The D1+D3CT fusion enzyme appears to have enhanced DNASE activity by RDA and may have more BT activity than equivalent mass of D1L3. Similarly, Mannose-6-phosphate glycosylation of the D1L3 enzyme (which is not evident either in vivo or in vitro immunoblots, and would not be predicted from sequence analysis) would be predicted to diminish BT activity.

Studies of Cell-free Nuclease Assays. While dissolved DNA is sensitive to D1 nuclease, most liposomal reagents protect DNA against D1 degradation. The former activity is here referred to as nuclease activity against “free” or uncomplexed DNA or RDA-nuclease activity or RDA activity. To compare the effects of D1 and D1L3s on free and FuGene™-6-bound plasmid, cell-free incubations were performed (FIG. 16). Free plasmid incubated in control media, which contains 10% serum, runs at a mixture of supercoiled and linearized. This effect is likely due to unspecified background nucleolysis or “nicking” the plasmid (perhaps trace active DNASE in serum). Media conditioned with D1L3 nucleases did not differ from control media, except that active D1L3 enhanced partial degradation into oligosomal ladders. Liposome-bound plasmid is generally protected against both D1 and D1L3, though results differ. After D1 exposure, all remaining plasmid migrated in a nicked or linearized conformation. After D1L3 exposure, mostly supercoiled plasmid is protected. A similar mass of supercoiled plasmid form appears to be protected in D1L3, D1L3 DT, and N196K-D3 exposure, despite the absence of a BT effect with the latter two mutant enzymes. Ultimately, D1L3-CM does not completely digest liposomal-bound DNA. These results suggest that cellular interactions are required to mediate the BT activity. These results do not clarify whether D1L3 binds or interacts with DNA-liposome complex before or after DNA or D1L3 has cellular interactions. The nature of the interaction remains undefined. D1L3 may attach to the cell surface prior to interacting with the DNA-liposome complex or be modified by cellular factors, or cellular events must first uncover DNA.

Summary of Known Sequence and Enzymatic Determinants of BT Activity. The previous results establish some sequence and enzymatic parameters that control the ability to confer BT activity. The highly basic D1L3 C-terminus is essential for mediating the targeting of the nuclease activity to lipofected DNA, and erecting a barrier to transfection (BT). Comparisons of known D1L3s predicted that a number of arginines or lysines, rather than a specific linear order of residues or motifs, is essential. These residues likely must be spaced from the SDH motif common to many nucleases by some small number of amino acids. In addition, these comparisons suggest similar protein or enzyme targeting may be achievable using small peptide elements similar to the D3CTs by virtue of the basic stretch. Examples of such recombinant DNASEs that could be used include, but are not limited to DNASE1, DNASE1L1, DNASE1L2, but may include enzymes of different character (ribonucleases, etc) and even non-mammalian provenance, as long as they can be added to cellular surfaces through diffusion through a liquid.

BT activity appears to require cellular interactions (i.e., exposure of lipid-complexed DNA to D1L3 conditioned media does not fully degrade plasmid). The inability of D1 and D3 pCT to confer BT strongly suggests that DNase activity, does not fully account for the BT effect. An additional capacity specific to the full-length D1L3 is needed. As discussed, D1L3 requires its Lys/Arg-rich C-terminus to provide BT.

Addition of the D1L3 C-terminus to D1 creates a novel chimera with BT activity. Polyarginine and polylysine are common components of lipid transfection reagents, where they are thought to enhance condensation around DNA. In addition, they may aid in intracellular translocation or nuclear translocation in endocytosis independent methods. Based on the cell-free incubation experiments, D1L3 does not appear to be able to fully degrade liposome complexed DNA, this suggests a cellular interaction of function to either activate the nuclease or allow access to the DNA.

The data suggests that the basic D1L3 C-terminus interacts with elements that are similarly targeted by liposomal polylysine moieties. Perhaps the D1L3 links to either the liposome-coated DNA or the endocytic vesicles via its C-terminus, but cannot not access and degrade the plasmid in cell-free media, until disruption of the liposome structure occurs. This represents a novel pathway for delivering enzymatic activity, specifically a nuclease, to cells.

An alternative hypothesis is that D1L3, rather than or in addition to blocking the access of the transfected DNA to the cells, may specifically target transfected cells for destruction; hence, DNASE1L3 may be used by macrophages or reticuloendothelial cells to kill cells that are permitting promiscuous uptake of exogenous DNA. This may be part of a biologic mechanism for the body to enforce clonal deletion of immune cells able to respond to autoantigens, and to destroy cells expressing exogenous, often infectious (for example, viral) DNA. Either hypothesis would explain why DNASE1L3 is a barrier to transfection, and would be consistent with a role in SLE prevention.

Expression of D1L3 and DNASE Activity in Macrophages. Media conditioned with 100 U/ml interferon-gamma (Ifn-γ) treated bone marrow-derived macrophages confers BT to HeLa cells (FIG. 29). The barrier to transfection is almost certainly not Ifn-γ itself, since media conditioned by HeLa cells treated with Ifn-γ or HeLa cells treated with equivalent doses of Ifn-γ neither confer nor possess a BT (FIG. 29). The agent(s) responsible for macrophage-secreted BT activity is likely and only D1L3. First of all, both D1L3 levels by anti-D1L3-peptide western and DNASE activity of a size consistent with D1L3 on zymogram are induced by Ifn-γ-treated macrophages (FIG. 22). Second, RDA-DNASE activity is induced in Ifn-γ-treated macrophage-conditioned media (FIG. 22).

Finally, the above in vitro experiments with media conditioned with transfected DNASEs prove that D1L3 alone is able to mediate this capacity.

D1L3 Levels and DNASE Activity in Lupus and Normal Macrophages.

Deficiency of deoxyribonuclease (DNASE) activity is postulated to predispose to the polygenic disease of systemic lupus erythematosus (SLE or lupus). SLE is a multifactorial disease characterized by autoantibodies against nucleosomal components, including DNA (Herrmann et al., (2000) Immunol. Today 21, 424-6). Among the hereditary factors suggested to predispose to SLE are defects in the clearance of DNA-associated antigens or immune complexed-antigens. For example, mutations in early complement factors, most prominently C1, are highly associated with SLE development in humans and mice (Walport, (2000) Nat. Genet. 25, 135-6; Botto et al., (1998) Nat. Genet. 19, 56-9). Complement forms is described as part of the “innate immunity” system. In addition, to aiding in the clearance of apoptotic remnants and immune complexes, the complement cascades play a role in inflammation and in the bodies response to pathogenic infectious agents.

This invention details the role in murine SLE of defects in “macrophage DNASE”, D1L3. The deficient activity of macrophage DNASE in the NZB/W F1 strain is associated with a defect in macrophage-conditioned BT activity associated with a paradoxical induction of D1L3 levels. Normally, this induction would be associated with a high Ifn-γ state, increased in macrophage-secreted DNASE activity and BT activity. The latter two are absent from SLE mice. The invention proposes a defect of D1L3 characterizes a model of polygenic SLE (NZB/W F1 mice); and suggests similar defects will be found in man. The invention describes a primary defect in D1L3 and a secondary defect in macrophage-secreted DNASE, (presumably D1L3). The invention asserts that the above defect is not due to DNASE1 deficiency, since there is no primary sequence variance in the coding sequence in SLE mice, and only mild defects of serum nuclease activity. In addition, DNASE levels in the urine, a compartment reflecting D1 expression, are equal (FIG. 25).

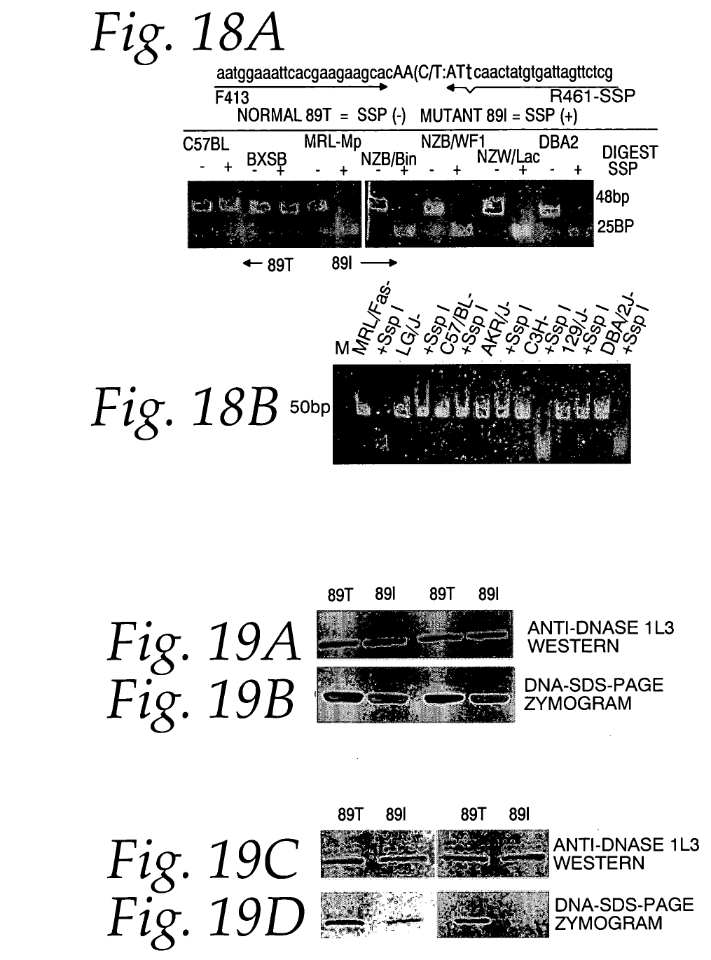

Primary Defect of D1L3 is Present in NZ and MRL Strains. The coding sequence of DNASE1 (D1) and the distinct macrophage-secreted homologue DNASE1L3 (D3) were analyzed in lupus-prone mice (MRL, NZ strains, and BXSB) and in non-SLE prone BALB-C and C57BL strains. The cDNAs spanning the protein coding sequence were sequenced and analyzed. No alterations that change amino acids were uncovered for DNASE1. A C to T transition at base pair 438 of murine D1L3 was present in both the NZ and MRL sequences (FIG. 17), and confirmed by analysis of amplified genomic DNA (FIG. 18). Thus, MRL and NZ models are homozygous for D1L3 alleles leading substituting threonine at residue 89 for isoleucine. The mutation encodes the non-conservative substitution of threonine at amino acid 89 by isoleucine (T89I) (FIG. 17), and will henceforth referred to as the 89I or mutant allele. The C57BL allele is referred to as the D1L3-89T or 89T or the wild-type allele. Similar terminology will be used to refer to the protein. By genomic PCR and/or sequencing, the 89I allele was also present in DBA/2, C3H, and A strains, and absent from the SM, AKR, LG, 129, BALB/C, BXSB, and C57BL.B10 strains. Genotyping of DNA from the BXD strains, obtained from Jackson laboratories, using the above PCR strategy showed complete concordance with markers D14Byu1 and D14Mit99, and the expected syntenic location on proximal chromosome 14 (data not shown).

Three a priori observations suggest 89I impairs D1L3's function: first, D3-89T is conserved across species (FIG. 17), thus, the 89I allele is described as mutant. Second, the mutation is adjacent to a highly conserved tyrosine involved in DNA-D1 contact (Jones et al., (1996) J. Mol. Biol 264, 1154-63) a potential PKC phosphorylation site (SRR) present in both D1L3 and D1. Finally, the residue nestles in a region of D1 involved in nuclease-actin interaction (FIG. 17) (Suck, (1994) J. Mol. Recognit. 7, 65-70). The NZ, MRL, C3H, and DBA/2 strains likely inherited this mutation from a common Castle-derived ancestor (Beck et al., (2000) Nat. Genet. 24, 23-5). However, the NZ and MRL strains are regarded as independent models, with generally non-overlapping susceptibility loci (Kono, (1999) in Genes and Genetics of Autoimmunity, vol. 1 (AN., T., ed.), Karger). While no murine SLE susceptibility allele matches this locus on mouse chromosome 14, a significant association was found for some human SLE patients to 3p14 region containing the D1L3 locus (Moser et al., (1998) Proc. Natl. Acad. Sci. USA 95, 14869-74). Ultimately, the selection of two “independent” SLE strains with the 89I mutation is highly suggestive by itself of a role for this allele in their genetic susceptibility to SLE.

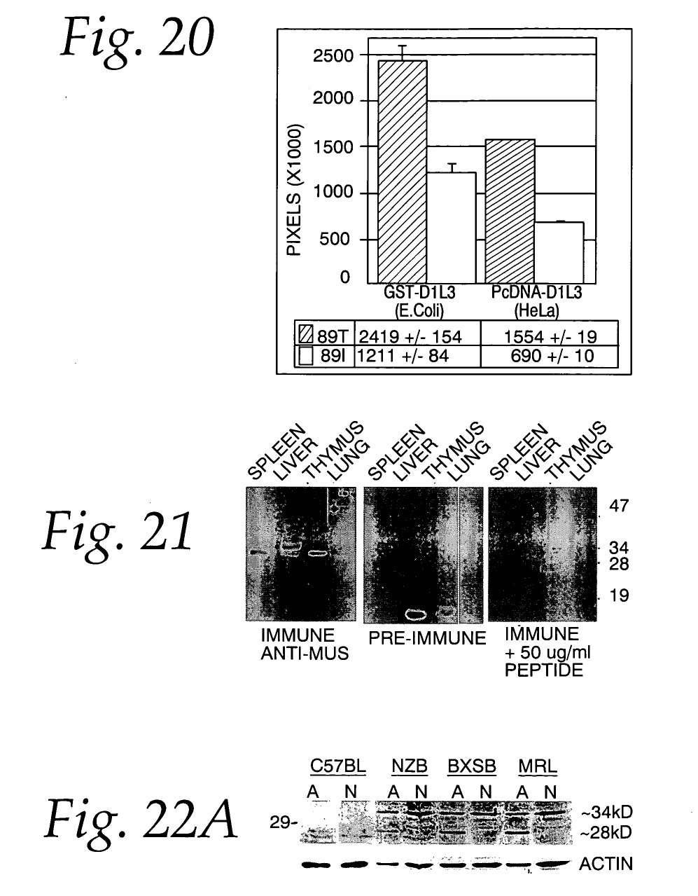

To determine if the 891- and 89T-D3 differed in nuclease activity after prokaryotic (E. Coli) and eukaryotic (HeLa) expression. First, DNASE activity in media conditioned by D1L3-transfected HeLa cells was measured by both zymograms (^{˜}28 kDa activity) and radial diffusion assays (RDAs) (FIG. 19). Media conditioned by pcDNA3.1 vector has nearly undetectable activity. Nuclease activity on zymograms was normalized relative to the immunoblotted D1L3 levels in cell lysates. 891 and 89T protein levels were equivalent. Zymograms from 891-transfected HeLa cells had 44% of 89T-transfected cells (n=16; p=0.0004 by Student's t-test) (FIG. 20). Similar results were observed when comparing RDAs of media conditioned by transfected cells and by similar assays transfecting D3-eGFP fusions immunoblotted with anti-GFP (N1-eGFP vector, Clontech) (unpublished observations). In conclusion, expression studies find partial loss of D1L3 function is present in the 89I enzyme.

In summary, when the activities of both wild type (89T) and variant (89I) D1L3 were assayed in vitro, the 89I D1L3 has about half the RDA-nuclease activity of the 89T enzyme (p<0.001). Thus a primary RDA-defect, albeit a limited one, of D1L3 is present in some models of polygenic SLE. Since the variant is also present in strains not know to be predisposed to SLE, it can be only one susceptibility factor, and additional secondary predispositions, which serve to decrease D1L3 activity may exists. Initial studies had shown undiluted conditioned media containing either 89I and 89T enzymes were no different in their ability to block liposomal transfection. However, when dilutions of the media were made, indications are that there may be a ten-fold difference between these two enzymes in BT activity (FIG. 31). This would be a powerful indictment of a significant primary role of D1L3 in 89I-associated murine SLE.

Additionally, FIG. 33 provides comparison of 89I and 89T BT activity. Compared by BT assay, the 89I enzyme is significantly defective. Normalized to immunoblotted D3 protein levels in pcDNA3.1-D3 transfected cells, 89I had a ^{˜}8 fold defect in BT activity (n=6, p-value=0.03 T-test). As shown in FIG. 33A, FD-nuclease activity in the conditioned media was similar in 89I and 89T, because of a nearly 2-fold higher level of 89I in transfected cell lysates. As shown in FIG. 33B, a ten-fold dilution of 89T nearly completely blocks GFP transduction, while liposomal gene transfection is possible in similarly diluted 89I-containing media. FIG. 33C provides relative levels of GFP expression for the 89T and 89I enzymes. Undiluted conditioned media in these experiments, which achieves higher levels of FD-nuclease activity than seen in Ifn-γinduced macrophage-conditioned media, conferred saturated BT activity with both 89I and 89T. These results establish that a significant primary defect in D3-BT activity is present in both the NZ and MRL strains of mice, and this finding in independent SLE models suggests a role in their susceptibility to the disease.

Normal D1L3 Expression in Tissues. Normal D1L3 protein is detected by anti-D1L3 immunoblot in spleen, liver, and thymus (FIG. 21). In both spleen and bone-marrow derived cells, expression of the 28 kDa cleaved product is present almost exclusively in adherent populations, especially cultured macrophages (FIG. 22A). This is consistent with other published observations (Baron et al., (1998) Gene 215, 291-301). The enzyme is inducible in macrophages by activating agents such as lipopolysaccaride (LPS), phorbol myristate acetate (PMA), and Ifn-γ (FIGS. 22B-D). Treatment of C57BL macrophages induces both levels of D1L3 in cell lysates but even more the levels of secreted DNASE activity (FIG. 22C). By zymogram, only a sharp 28 kDa band of activity consistent with D1L3 is observed and only after Ifn-γ stimulation.

Abnormalities in D1L3 Levels and DNASE Activity in SLE Mice. In cultured macrophages and leukocytes from C57BL mice, secreted nuclease activity correlates with the expression levels of the Ifn-γ-inducible D1L3. Therefore, initially the nuclease activity by RDAs and the levels D1L3 enzyme by western in SLE and C57BL mice were examined. Young mice (<12 weeks old) were studied in all cases. While the DNASE activity expressed by cultured leukocytes from lupus mice is similar to that found in C57BL mice, this is only achieved by a 4-5 fold level of D1L3 protein induction in splenocytes and 2-3 fold induction in macrophages. In the MRL and NZ models, this deficiency likely reflects in part the presence of the hypomorphic 89I allele, but secondary defects are likely involved in these models and in the BXSB strain, which has a wild-type D1L3 allele. Deficiency in D1L3 activity is predicted to predispose mice to lupus-like disease by interfering in degradation of DNA-containing antigenic material. Finally the results in this section only refer to RDA-nuclease activity. The RDA nuclease activity describes the enzymes activity against “free” DNA. D1 only shows RDA nuclease activity and minimal, if any, BT activity. D1L3 has both, though appears to have less RDA activity than DNASE1. In a following section, a defect in BT activity in media conditioned by macrophages from SLE mice is described. That activity is dependent but not identical with RDA activity as shown in prior sections. The table in FIG. 23, represented in graphic format in FIG. 28, summarizes the pixel values observed for RDAs and immunoblots of the various compartments. All the measurements described in this section refer to this table.

The DNASE activity in urine, which expresses high levels of DNASE I, was analyzed (Takeshita et al., (1995) J. Biochem. (Tokyo) 118, 932-8; Nakajima et al., (2000) Exp. Clin. Immunogenet. 17, 71-6), and no differences among the strains/models studied was found (FIGS. 24 and 23). Urine had higher concentration of DNASE of all the compartments examined. In urine, there appeared to be few discrepancies in DNASE activity between lupus mice and C57BL. This supports the contention that DNASE I likely does not play an important role in polygenic SLE.

DNASE I appears to be the main serum DNASE. Previous mouse studies found serum DNASE deficiency in NZ mice, and attributed this to D1 defect (Macanovic et al., (1997) Clin. Exp. Immunol. 108, 220-6). In addition, D1 −/− mice ablate nearly all DNASE zymographic activity (Napirei et al., (2000) Nat. Genet. 25, 177-81). While D1L3 can be detected in serum by immunoblot in both mice and humans, zymograms i bands of DNASE activity are likely DNASE I-related (>30 kDa) (FIG. 25). There are differences of uncertain significance in the patterns of serum DNASE activity by zymogram and levels of activity between SLE and wild-type mice. The 89T lupus-prone BXSB mice had more activity by RDA and zymogram (FIG. 23) than either NZBW or MRL (p<0.001). While serum D1L3 levels in NZ mice were less than those in MRL (p>0.05), serum RDA activity in NZBW was not significantly different than wild-type (Macanovic et al., (1997) Clin. Exp. Immunol. 108, 220-6).

The most striking observation was D1L3 induction in splenocytes (4-5 fold) (FIG. 26) and macrophages (2-3 fold) (FIG. 27) of all lupus models relative to C57BL (p<0.01) (FIGS. 23 and 28). Similarly induced levels were seen in control (C57BL) macrophages after treatment with PMA or Ifn-γ, but unlike lupus mice, induction led a parallel increase in secreted DNASE activity. In contrast, RDAs of media conditioned by SLE splenocytes and bone-marrow derived macrophages failed to show increased activity. Thus, relative to expression levels, macrophages and splenocytes from both 89I and 89T SLE strains show a defect in DNASE activity. In macrophage-conditioned media, the RDA activity of the 891 strains was significantly lower than C57BL (p<0.01). An additional observation was that the 89T BXSB mouse, while still relatively deficient, showed significantly more activity than the 89I lupus mice (p<0.01) (FIG. 27).

The data supports hypothesis in which murine models and patients with SLE and normal D1 enzymes, the lupus-predisposing deficiency of DNASE activity is caused by defects in macrophage D1L3. Despite induction, the lupus mice fail either in secretion or have inhibitors of the enzyme. On fresh isolation, lupus macrophages appear to be in an Ifn-γ induced but D1L3 impotent state. It is possible that the secondary defect in DNASE activity seen in SLE mice is due to aberrant cytokine milieu, leading to macrophage malfunction. It is also possible that D1L3 malfunction is crucial in dendritic (antigen-presenting cells) or lymphocytes. While D1L3 is clearly expressed by macrophages, hence a D1L3 defect in this lineage is the prime candidate for causing the susceptibility, the defect could be due to the defect in another cell line.

While 89I lupus strains have significantly lower macrophage DNASE activity than 89T BXSB and C57BL, all lupus strains, including the 89T BXSB, have defects of similar magnitude relative to the protein levels (FIGS. 23 and 28). Thus, the 89I mutation is likely not the only cause for the DNASE defect in SLE mice. In addition, 89I is present in strains not prone to spontaneous SLE such as DBA/2, A, and C3H. At a molecular level, the observed in vivo defect in relative activity is greater than that expected from 89I allele. Ultimately, the relative DNASE deficiency in SLE mice must have a secondary (non-allelic) component.

The full cause of the relative D1L3 activity defect in SLE macrophages is unknown, especially in the 89T BXSB strain. The induced cellular expression suggests a failure of secretion or the presence of inhibitors. No D1L3 inhibitors were apparent in RDAS from serum mixing experiments between normal and SLE mice (data not shown). Previous studies found anti-D1 antibodies capable of inhibiting D1 activity in MRL mice (Madaio et al., (1996) Eur. J. Immunol. 26, 3035-41); however, this would not explain a deficiency in media conditioned by macrophages.

Macrophage numbers appear expanded in SLE (Muller et al., (1991) Eur. J. Immunol. 21, 2211-7). The cellular correlate of macrophage DNASE defects in SLE is hypothesized to be deficient degradation of phagocytosed material. Defects in macrophage phagocytosis have been described (Licht et al., (2001) Lupus 10, 102-7; Laderach et al., D., (1998) J. Leukoc. Biol. 64, 774-80), but these results are controversial (Russell et al., (1986) J. Leukoc. Biol. 39, 49-62). Clearance of immune complexes, defective in C1q deficient mice, may be a better assay of SLE (Nash et al., (2001) Clin. Exp. Immunol. 123, 196-202; Davies et al., (1992) J. Clin. Invest. 90, 2075-83). Ultimately, the BT assay may be even better at determining who is predisposed to SLE. In SLE mice, macrophages appear activated due to abnormal levels of circulating cytokines (Alleva et al., (1997) J. Immunol. 159, 5610-9); this state could be responsible for the induced levels of D1L3 in SLE mice. D1L3 is inducible in normal macrophages by activators such as Ifn-γ.

Splenocyte and macrophage D1L3 protein levels are induced in polygenic murine lupus relative to C57BL. In addition, the 89I DBA/2 strain, and not the 89T C57BL, SLE can be experimentally induced by the exposure to an extrinsic anti-idiotype manipulation (Mozes et al., (1997) Clin. Immunol. Immunopathol. 85, 28-34). Thus likely other susceptibility factors in 89I SLE mice either increase the production or circulation of DNA-associated auto-antigens, perhaps by overactive apoptosis, or that additional factors decrease nuclease activity further. For example, inhibitory autoantibodies to D1 may play this role in MRL mice (Madaio et al., (1996) Eur. J. Immunol. 26, 3035-41).

These observations suggest that introduction of the 89I allele on to the 30) genetic background of lupus-prone congenics derived from NZ×C57BI crosses (Morel et al., (2000) Proc. Natl. Acad. Sci. USA 97, 6670-5) may exacerbate disease. In addition, the 89I allele may have synergistic lupus-promoting effects with other defects that potentially alter antigen clearance such as murine D1 (Napirei et al., (2000) Nat. Genet. 25, 177-81), C1q (Botto et al., (1998) Nat. Genet. 19, 56-9), If n-γ receptor knockouts, or SAP null (Bickerstaff et al., (1999) Nat. Med. 5, 694-7) mice. Experimental induction of nucleosomal autoimmunity with pristane or other agents may be associated with increased cellular levels of D1L3 protein in 89I strains not prone to spontaneous lupus, such as DBA/2 and C3H. Finally, based on linkage data in human SLE (Moser et al., (1998) Proc. Natl. Acad. Sci. USA 95, 14869-74), human patients with SLE are predicted to also have primary or secondary D1L3 abnormalities.

In conclusion, primary and secondary defects in the activity of this leukocyte DNASE are associated with polygenic lupus-prone strains of murine SLE. DNASE1L3 may have the foremost role among nucleases in suppressing SLE. Serum RDA activity may not be the most important requirement for lupus-prevention, and may explain why systemically administered D1 appeared to be ineffective as therapy of the disease in the NZB/W F1 model and humans. D1 may only be part of the organism's defense against nucleosomal antigen stimulation, and D1L3, a circulating actin-resistant macrophage-produced DNASE may be the relevant enzyme to supplement in lupus.

BT Activity in Lupus and Normal Macrophages. Ifn-γ-dependant induction of D1L3 levels and secreted DNase in normal macrophages was demonstrated above (FIG. 22); as expected, this induction is associated with increased BT activity. Media conditioned by Ifn-γ-stimulated C57BL bone marrow-derived macrophagesis confers a barrier to liposomal transfection to HeLa (FIG. 29). Increased BT activity was obtained with higher doses of Ifn-γ (1000 U/ml vs 100U/ml) (FIG. 2B). In the absence of Ifn-γ stimulation, macrophages from C57BL, which have low baseline D1L3 levels, had weaker BT activity. Anti-D3 immunoblots on cell lysates for this experiment did not show as a parallel linear induction (FIG. 22C), suggesting that part of the increased DNASE activity is due to secretion or other enhancements of already synthesized protein.

If SLE mice were deficient in D1L3 activity and D1L3 was the sole source of BT activity, macrophage-conditioned media from SLE mice would have deficient BT activity. Preliminary experiments comparing MRL and C57BL macrophages show no difference in BT activity, yet these results were obtained after 7 day in vitro culture in GM-CSF (data not show). These experiments suggest that MRL macrophages can recover from the D1L3 defect. When media is conditioned for 36 hours by freshly isolated macrophages from NZB/W F1 mice versus C57BL, a qualitative difference in BT activity is observed (FIG. 29). Conditioned media from C57BL macrophages, but not NZB/W F1 hybrids blocks transfection. Serum from these mice did not differ in BT activity; 10% fresh serum has BT activity for both mice, despite the lower RDA-nuclease activity found in NZB/W F1 serum (FIG. 28).

In conclusion, macrophages from SLE-prone NZB/W mice are deficient in BT activity (FIG. 29). It appears that this paradigm is common to polygenic SLE in humans, since in all murine models there are macrophage DNASE1L3 defects both in the presence or absence of a primary defect in DNASE1L3. The following predictions would follow from this discovery:

(1) Primary defects in DNASE1L3 are likely present in some humans with SLE and form part of their susceptibility to the disease

(2) Secondary defects in DNASE1L3 BT activity are present in some humans with SLE and form part of their susceptibility to the disease

(3) Enhancement of DNASE1L3 activity in the appropriate compartment may prevent development, block progression, or possibly treat SLE. It may be possible to address the above goals by increasing serum concentrations of active enzyme by providing this or other enzyme combinations with BT activity by either intravenous or other delivery systems. In mice, this may be achieved via intraperitoneal injection or by other systemic or local delivery systems. The enzyme may also need to be delivered to appropriate tissue sites that are normally addressed by macrophage secretion.

(4) If D1L3 defects predispose to SLE and a major biologic role of D1L3 is a BT activity, then a potential side-effect of repeated courses of in vivo liposomal gene therapy could be SLE-like autoimmunity.

(5) Finally, testing for a defect in BT activity may predict which individuals are at risk for SLE. In mice, the assay was described using bone marrow macrophages; these are not easily obtained from humans. However, human D1L3 is detectable by anti-D1L3 immunoblot in human serum and peripheral mononuclear cells from normal and SLE patients. One possible assay would be the in vitro culture of such a sample derived via peripheral phlebotomy, in similar culture media to that used above, and sampling of the ability of the Ifn-γ induced conditioned media to confer a barrier to liposomal transfection. It may not be necesarry to obtain using a purified sample of macrophages, and the monocyte-derived populations from the periphery may suffice.

(6) An alternative assay may test the in vivo ability of individuals to block an administered liposomal transfection “load” in vivo. The proposed test of the in vivo transfection susceptibility would require the administration to a live subject of a D1L3-susceptible marker plus reagent, for example, but not limited to a plasmid marker plus liposomal reagent or an adenoviral gene transduction reagent. The measured variable would be either the distal recovery of the administered agent or a measure of the expression of the transduced agent. For example, it would be predicted that a systemic D1L3 defect would be accompanied by an increased ability to transduce liposome-mediated DNA markers. In the present example, a non-allergenic soluble and secreted gene product could be used as a DNA marker, for example, an individual would be administered by intravenous route with a plasmid encoding a foreign but non-immunostimulatory peptide or protein. The protein could be an allelic variant of a normal protein, which does not elicit an immune response and yet can be distinguished from the endogenous product by either ELISA, or in vivo, ex vivo, or in vitro functional assays, or by immunodetection. For example, a protein or hormone which can transiently alter metabolic or biochemical parameters can be administered, and the measure of BT becomes the ability of the transduced gene to alter the predicted biochemical parameter. The present model predicts that individuals predisposed to SLE because of D1L3 defects will allow greater levels of gene transduction to occur.

Analysis of DNASE1L3 sequences in human SLE. To determine if mutations potentially leading to D3 deficiency were present in human SLE, DNA samples were obtained from the Lupus Multiplex Registry & Repository (LMRR) (http://omrf.uokhsc.edu/lupus/). This NIH-sponsored study maintains data, serum, and DNA from families that have at least two (2) well-characterized members that have been diagnosed with lupus. These samples were also of interest because they had shown a significant multipoint linkage to the 3p21 D3S1766 microsatellite marker with a LOD score of 1.68 using a recessive model (Moser et al., (1998) Proc. Natl. Acad. Sci. USA 95, 14869-74). Dr. Harley, LMRR director at the OMRF (Oklahoma Medical Research Foundation), kindly enriched the DNA samples in our possession with individuals, mostly African-Americans, showing linkage. D3S,1766 and DNASE1L3 showed no recombination in radiation hybrids.

The genomic sequence contained in locus NM_{—}004944 and our previous studies suggest that the gene encoding the ^{˜}1 kb D3 transcript spans approximately 15-20 kilobases. Genomic fragments encompassing all eight D3 exons and the immediate (10-12 bp) peri-exonic sequence (using the primers in Table 1) were amplified using PFU/TAQ polymerase mixture for greater fidelity. The fragments were purified with MinElute PCR purification spin columns (Amersham), and subjected to bi-allelic dideoxy fluorescent sequencing with the same primers used for amplification. Forward and reverse sequencing was performed using a 96 well format at the SIU Soybean Genome Center at Carbondale in 96 well format (www.siu.edu/^{˜}pbgc/). Sequences were analyzed and edited with DNASTAR SeqmanII (Lasergene).

Over 95% of the stated D3 sequence for 50 affected individuals and 33 unaffected relatives (^{˜}7500 bp) from 29 independent pedigrees has been obtained with over 90-95% certainty. Exon 4 is being resequenced with internal primers due to sequence ambiguities over a portion of the exon. DNA is available for an additional 48 unaffected relatives from these pedigrees. Heterozygous changes were ascertained only when the alteration was evident on both strands. To date, analysis has identified seven variants (Table 2).

TABLE 1

Human genomic DNASE1L3 sequence and primers

| | | | |
|---|---|---|---|
| NM_004944 | NT_005670 | Forward primer | Reverse primer |

| | | | | |
|---|---|---|---|---|
| 1 | 1-15 | | | |
| 2 | 15-213 | 2236149-2236345 | CAGCACTCCAAGCACTGCTGTC | AAGTCTGCAGACAGGAGAGAGG |
| 3 | 214-300 | 2238805-2238893 | TCTGTGGTAAATGACCTCAAAC | TGTGTGAACTGGTGGTCAAGTG |
| 4 | 299-391 | 2241551-2241640 | GAATGTTTACTCTTCAAGATGCAGT | CGATTTATTGGTGGCCATGTTCC |

| | | |
|---|---|---|
| Internal primers (exon 4) | TACTCTTCAAGATGCAGTGTT | ATTGGTGGCCATGTTCCAGG |

| | | | | |
|---|---|---|---|---|
| 5 | 391-502 | 2242230-2242342 | GCTGACTCAGCTGGGAGGACTT | TTAATGGGCTCATGCTCAAGGCT |
| 6 | 503-615 | 2246002-2246114 | AGTGGTTTATGAGACCTTGACAG | TGCTAGATGGGAATTCGTCTGAC |
| 7 | 615-771 | 2249133-2249290 | CAGTGTGAAAGCATTCTGCAGCA | GCACACCAAGCACTGTGGTGAG |
| 8 | 772-869 | 2253677-2253775 | CTTGGTTTGATCTCAGCAATCAC | ATCTTTCCAGGACAATGGCATAG |
| 9 | 870-1079 | 2254310-2254463 | GGCATCTCCTAATTTCCATGTCT | CCTTCCAATTTGGCTCAAGTCAG |

TABLE 2

| | Nucleotide | | |
|---|---|---|---|
| | NM_004944 | Alteration | Presumed effect |
| 1 | 2246063 | C > G | Missense-Ile166Met |
| 2 | 2246069 | G > A | Silent-Glu168Glu |
| 3 | 2241596 | G > C | Silent-Arg92Arg (exon 4) |
| 4 | 2253754 | A > G | Missense-Tyr > Cys at aa 261 (Y261C) |
| 5 | 2254337 | T > G | Missense-Val277Gly |
| 6 | 2246119 | G > T | Affects splice donor consensus IVS6 + 5 |

The missense changes and the splice donor mutation were further evaluated. All missense mutations were first confirmed by re-amplification from original DNA sample, cloning of amplicons, and sequencing of 6 independent clones (FIG. 34). In addition, when possible, the genotype of other members of the pedigree was examined. The I166M mutation is not being evaluated further since pedigree analysis shows that it is part of the haplotype containing the Y261C and Glu 168Glu variants. All three variants are present in two unrelated individuals, one affected and the other unaffected. The affected woman with the Y261C mutation inherited the mutant IVS6+5 T allele on her other allele from her mother, and the Y261 C from her father. Thus she is a compound heterozygote for two different mutations; while the unaffected woman carrying the Y261 mutation is homozygous for the more common and consensus IVS6+5G allele. The effect on activity of the Val277Gly variant found in one patient remains to be analyzed.

To analyze the Y261 C mutation, this mutation was inserted by mutagenesis into a pcDNA-D3-HA vector, which expresses the full-length human DNASE1L3 fused to hemagglutinin at the C-terminus. The Y261 C enzyme has complete loss of function by zymogram, RDA, and BT assay (FIG. 35). This was not unexpected: the mutation alters a highly conserved tyrosine and likely distorts the structure by creating a novel uncoupled cysteine in an extracellular protein predicted to contain two disulfide bonds. Since the affected individual is a compound heterozygote for Y261C/IVS6+5 G>T, this patient may be a complete loss of function. However, it serves to recall that the only reported DNASE1 mutations in human SLE are heterozygous. The finding of an independent loss of function mutation in D3 in association with the same phenotype in man and mouse serves as compelling evidence that D3 deficiency increases SLE susceptibility.

The biologic significance of the mutation (or polymorphism) at IVS6+5 G>T (or G t T at the +5 nucleotide of the splice donor consensus just after exon 6) has not been established (FIG. 35). It was remarkably common in our African-American pool of SLE (41% of alleles and present in ^{˜}55% of patients (FIG. 14). The +5G of the intron is conserved in >85% of splice donor sites; however, this does not imply that a splice donor site with +5G mutates in 15% of alleles; it states up to 15% exons are not +5G. The most common allele in our population, and likely normative in other populations, contains a G at this site; thus the T allele is very likely to be a mutation, and hence could be deleterious. The final effect of intronic mutations in residues other than the immediate 2 residues to the junction (+/−1 and 2) are difficult to predict a priori. Numerous disease-related mutations in diverse genes affect residues at a similar intronic position, including for diseases such as, but not limited to: thalassemias (Danckwardt et al., (2002) Blood 99, 1811-6), Usher syndrome (Bolz et al., (2002) Nat. Genet. 27, 108-12), phenylketonuria (PKU) [http://data.mch.mcgill.ca/pahdb_new/about.html, 2002], and cystic fibrosis (Highsmith, et al., (1997) Hum. Mutat. 9, 332-8; Bisceglia, et al., (1994) Hum. Mutat. 4, 136-40). The alterations usually result in intron skipping, with attendant frame-shifted or insertion product or alternatively lead to a decrease or absence of expression. The compact and highly folded DNASE proteins are unlikely to tolerate any significant stretch of amino acids. The association and linkage of the mutation to SLE will be further studied by examining the enzyme levels and D3 activity in leukocytes from IVS6+5T homozygotes, and by examining RNA from such patients for abnormal transcripts or expression.

When control populations are compared for the prevalence of the intronic +5 T allele, the following prevalence data is found for the T allele: 41% of alleles in the studied SLE population; 7% of alleles in a random sampling of Springfield, Ill. (^{˜}90% Caucasian); 25% of alleles in random African American controls. This suggests that the T allele is more frequent in the African-American population. This may be consistent with the LMRR linkage data for 3p21 and imply that this is a strong susceptibility at least in this population.

Since our studied SLE population derived from 26 pedigrees, analysis of the samples in the following fashion showed these results: If each pedigree was counted as a “proband”, the allele frequency of T was 41%; when compared to the random African-American value of 25%. A p-value of <0.5 would require a chi-square value of 3.7 for 1 degree of freedom, yet a value of 2.75 is obtained. If the above numbers are converted to alleles, then a chi square of 5.6 with a p value of 0.02 is obtained. This strongly suggests that if the observed pattern of allele distribution is found in a larger population of affecteds, then significance would be achieved without recourse to pedigree assignment.

Ultimately, these findings confirm the hypothesis that loss of function mutations or polymorphisms of DNASE1L3 are present in human SLE.

They suggest that in polygenic human SLE, these mutations may be common. These findings suggest that sequencing or determination of in vivo DNASE1L3 activity in humans may help diagnose or predict susceptibility to SLE. Finally, if deficiency is associated with SLE, the restoration of DNASE1L3 activity in human SLE may mitigate or prevent the disease.

Experimental Materials and Methods:

Murine Stocks and Genotyping. Mice that were 8-weeks-old of C3H, DBA/2, BXSB, C57BL/6, and MRL strains, as well as the NZB/W F1 hybrid were studied. Purified DNA for PCR based genotyping was obtained from LG, AKR, SM, 129, A, BALB/c, and from 25 BXD (C57BL/6×DBA/2) lines (Jackson Labs). Strain characteristics, including BXD marker data, were obtained from the MGI 2.6 database at http://www.informatics.jax.org/.

For sequencing of D1L3 cDNAs, total liver RNA was isolated using a guanidinium-phenol method (TRIZOL; Lifetech), and PCR-amplified first with D3F9 5′ GCACTGTCTTCATCCAGCCTG and D3R10 5′ CTTAAGGCCTCGCACTCTGGAT, then sequenced with D3F10 5′ CCACCACTGCAAAGATGTCC and D3R9 5′ CTTCTGACATCGAATTTGAGT. For D1, cDNA first amplified from kidney total RNA with D1F128 5′ CTGCTGCAGCCGTCTCAGATTG and D1R1029 5′ AAGCAGTATGGCTGAACTGCTC; and sequenced with the same primers. The CDNAs were sequenced by ABI sequencers at a core facility (Iowa University Sequencing Facility), and analyzed initially by BLAST comparisons with Genebank sequences.

The genotype at residue 438 was analyzed in multiple strains by amplifying a 48 bp fragment surrounding this residue from genomic DNA with the following primers, F413 and R461SSP, which mutates residue 441 from A to T (FIG. 18). The primers flank residue 438 (italicized in the underlined SSP1 site AATATT)(FIG. 18). The amplified mutant fragment is susceptible to SSP1, while the normal sequence is resistant to cleavage. Products were analyzed by 1×TAE 8% gel (acrylamide/bis 30:1) electrophoresis using mini-Protean II gel apparatus (Bio-Rad).

Plasmid Constructs. Full-length mice D1L3 cDNA was amplified from C57BL liver RNA with the SuperScript II system (Life Tech) with addition of 0.1 U Pfu polymerase (Stratagene) using primers F575 GCAGAGCTGGTTTAGTGAACCGTC 3′ and D3-Kpn GAGCGTGGTACCTAGGAGCGATTG 3′. The amplified cDNA was cloned into BamH1/Kpn 1 digested eukaryotic expression vector pcDNA3.1 (Invitrogen). Both strands of the amplified products were directly sequenced using dideoxy ABI sequencers. The full-length D1 cDNA was similarly amplified from kidney RNA using D1F128 CTGCTGCAGCCGTCTCAGATTG 3′ and D1R1029 TTCGTCATACCGACTTGACGAG 3′ and after initial subcloning into a TA vector, Xho1 and Kpn1 flanked construct was cloned into pcDNA3.1.

The sequences of both the D1L3 and D1 clones were identical to reference sequences for the mice genes (for DNASE1L3 accession # NM_{—}007870 and for DNASE1 accession # NP_{—}034191.

The D3 pCT enzyme was made by mutagenizing Ser289 (TCA to TAA) with a stop codon to truncate D1L3 C-terminus (QuikChange XL, Stratagene) using primer D3p289-310 AGCTACAGTCTTAAAGGGCCTTC 3′ and its reverse-complement, and change confirmed the mutation by sequencing.

The N196K-D3 cDNA clone was created by point mutagenesis using primer GGTGATTTCAAgGCCGGCTGTAGCTA 3′ and its complement.

The mutation was confirmed by the creation of a novel Hae3 site in the cDNA. The human N191K clone was created by mutagenesis of the wild-type (wt) EST 82269 (Rodriguez et al., (1997) Genomics 42, 507-13). Both wt and mutant (N191 K) cDNAs (starting at bp 53) were cloned in frame to C-terminus of GST protein (PGEX4T3). The N191K mutation was PCR amplified with a primer carrying an internal single bp change, and recloning fragment into the wt-cDNA.

To create the fusion of N-terminal D1 to the C-terminal (289-305) D1L3, the pcDNA3.1-D1 plasmid was mutagenized using primer DNase1-BGL2F/R to create an artifactual BGL2 site centered at the next to last codon. The single BGL2 site located in a non-functional sector of pcDNA was mutagenized to abolish the restriction site. Next, the terminal ˜60 bps (289 to past poly-A tail) of the D1L3 were amplified with D3-CT-Bam F and M13F, creating a novel upstream BAMH1 site. This Bam-Kpn fragment was cloned into Bgl2-Kpn digested pcDNA3.1-D1-BGL2 (FIG. 14).

To compare the in vitro activity of 89I and 89T enzymes, D1L3 cDNA (AA 11-305) was cloned in-frame downstream of a prokaryotic GST cassette (pGEX4T) (Amersham Pharmacia). Oligonucleotide site directed mutagenesis was used to synthesize the mutant 89I allele using the QuikChange kit (Stratagene) and the primers MRL-F and MRL-R. For cloning into pcDNA3.1, the primers F575-GFP GCAGAGCTGGTTTAGTGAACCGTC 3′ and D3R13-Kpn GAGCGTGGTACCTAGGAGCGATTG 3′ were used to amplify D1L3 and introduced into pcDNA3.1 (Invitrogen) after Nhe1 and Kpn1 digestion. Mutant counterparts of the pcDNA clone were created with QuikChange kit. Vector inserts were subjected to single strand sequencing for confirmation.

Transfection Reagents and Resistance Assays. Lipofection of respective pcDNA3.1-D1 or -D3 (2 jgs purified with Promega Wizard Plus DNA system) was performed independently into 1-2 million HeLa cells, which produce a low background of secreted nuclease activity, when grown in DMEM with 10% FCS (LifeTech) in 6 well culture plates (Becton-Dickinson). No difference was observed in cell morphology between control, D1 and D3 transfected or exposed cells. Media of the transfected cells were conditioned for 36-48 hours and served as source of the respective enzymes. Nuclease activity in conditioned supernatant was measured by RDAs, zymograms and by testing activity against supercoiled pBluescript (Stratagene).

To test for the ability of the nucleases to block transfection, the media in individual wells of naive HeLa cells were replaced with conditioned media, and cells transfected with 2 gs of N1-eGFP plasmid (Clontech) using various transfection reagents (FIG. 11) and their respective protocols. GFP expression by immunoblot directly correlated with transfection efficiency in present experiments. Thus, transfection efficiency was compared by densitometric analysis of GFP immunoblots.

GFP expression was also examined by fluorescent microscopy using a FITC filter. For microscopy, cells were fixed for 5 minutes in 4% paraformaldehyde dissolved into 1% PBS.

Reagents. Recombinant mouse GM-CSF and IL-4 were obtained from R&D Systems. Ifn-γ (Genzyme) was kindly donated by Dr. Dennis Crouse (SIUSOM). Chemicals and enzymes, including Taq polymerase, were from Fisher Scientific (St. Louis) and Promega (Madison). Deionized water further purified with a Millipore Milli-Q system (Millipore) was used.

Antibody Reagents and Immunoblotting. After 24-36 hours, cells transfected with N1-eGFP were lysed in SDS-buffer and immunoblotted for expression with anti-GFP at 1:5000 dilution (Clontech). To confirm nearly equal protein loading per lane, anti-β-actin monoclonal antibody at 1:7,000 dilution (Sigma) were prepared. For immunoblots, 100 mg protein was eletrophoretically separated by 12% SDS-PAGE gel, followed by electrotransfer to Immobilon-P membranes (Millipore) for expression studies. Immunoblotting was performed using ECL detection (Amersham).

Transfection efficiency was compared, wherever possible, by pixel densitometry of the scanned images using Scion Image 4.02 software.

Student's T-test was used to compare paired results.

Rabbit antisera were raised against the KLH-linked mice D3 peptide sequence: KAYDLSEEEALD (Sigma-Genosys). The immunizing peptide represent hydrophilic regions specific for DNAS1L3, and not other DNAS1Ls. The hD3 peptide KAYDLSEEEALD was used to develop rabbit anti-human D1L3 specific antisera, the sera was tested by the ability to recognize by western the expected 34 and 29 (signal peptide cleaved) kDa bands in both serum, peripheral leukocytes, and human D1L3 transfected cell lines (FIG. 32), the absence of this recognition in pre-immune sera, and the ability of the above peptide to block the reaction (FIG. 12B) has been found to be useful in developing antisera in rabbits against the human protein. The specificity of the polyclonal antisera was demonstrated by the ability to recognize by western the expected 34 and 29 (signal peptide cleaved) kDa bands in both tissues and D3 transfected cell lines, the absence of this recognition in pre-immune sera, and the ability of the above peptide to block the reaction (data not shown). Secondary HRP-linked antibodies were used as per ECL protocol (Amersham Pharmacia). Monoclonal anti-GFP were obtained from Clontech and anti-b-actin antibodies from Sigma.

Secondary HRP-linked antibodies were used as per ECL protocol (Amersham Pharmacia). Cell lysates for immunoblots were obtained by boiling in 2% SDS buffer. Per well, 100 ug of protein was electrophoretically separated by 12% SDS-PAGE gel (acrylamide/bis 30:1) at room temperature and 100 V for 3 hours using mini-Protean II gel apparatus, followed by electrotransfer to nitrocellulose (Sigma) in tissue studies, or Immobilon-P membranes (Millipore) for cell expression studies. Equal loading of protein samples was determined by BCA Protein Assay (Pierce) and/or immunoblot of cellular actin expression levels. Membranes were blocked for 2 hours in PBS-0.1% Tween-20 (PBS-T) containing 5% non-fat dry milk, 4% goat serum, and then incubated overnight at 4° C. with PBS-T containing 5% dry milk and either anti-D3 antisera at 1:4000 or the anti-GFP at 1:2000. Following one 30-minute and three 15-minute washes in PBS-T, membranes were incubated with peroxidase-conjugated anti-rabbit antibody at 1:5000 (Amersham Pharmacia) for 1 hour at room temperature. After three 15-minute-washes with PBS-T, ECL detection was performed with X-ray films (Fisher Scientific). Images and photographs were scanned as 600 dpi Tiff images, and analyzed by measuring pixel area and intensity of traced bands, using Scion Image 4.02 software for PC (http://www.scioncorp.com/).

Nuclease Assays. Nuclease activity against “naked” or free DNA in CM was quantified by densitometry of RDAs. For assays of media by murine cells, RDAs were performed using conditioned media, after 36 hour incubation of equal numbers of target cell population. Each conditioned media was sampled in triplicate. RDAs for all four mice were performed on a single dish. For RDAs, 2-5 ul of conditioned supernatant was dotted on 100 mm dishes containing 2% agarose, 250 ug/ml DNA, 0.001% EtBr, 0.5% filtered milk, 10 mM Ca⁺²/MgCl₂; and 12.5 ug/ml kanamycin. Multiple observations were generated for every sample obtained. After 24 hours of 37° incubation, plates are photographed under UV transillumination; and scanned photos were analyzed with NIH image. For RDAs, the activity was quantified as the product of mean density and area (traced on image) of zones of clearing. For observations of serum/urine, 2-5 ul of serum were dotted on agarose plates.

Zymograms are in-gel renaturation assays performed by SDS-PAGE through a gel impregnated with 200 g/ml DNA and ethidium bromide, eluting away SDS with repeated washes in 25 mM Tris-HCl pH 7.5, then incubating for 12-16 hours at 37° C. in 25 mM Tris-HCl pH 7.5 with 10 mM Ca⁺²/Mg⁺² buffer. Activity is measured as dark “bands” of DNA digestion (Rosenthal et al., (1977) Anal. Biochem. 80, 76-90) and quantified using scanned images using NIH Image. Photographed RDA results were scanned 600 dpi Tiff images, and activity was measured as pixel area times density of traced zones of clearing (n=3 for each zone). Student's T-test was used to compare paired results. Nuclease activity ratios were obtained by comparing intra-plate results. RDA activity of control and N196K media was faint and comparable to the activity of unconditioned culture medium.

Cell-Free Nuclease Activity Assay. Free plasmid DNA or plasmid complexed with lipofection reagent was incubated with conditioned at 37° C. for 1 hour. The incubation was terminated with 50 mgM EDTA, and DNA extracted using Promega Wizard DNA Purification System, eluted in 40 l water, and electrophoresed on a 1% agarose gel.

Prokaryotic Expression and GST Purification. Human D1L3-GST expression was performed by growing transformed BL21 Cells (Stratagene) at log phase were induced in 50 mg/ml IPTG for 5-7 hours at 32°. Bacterial pellets were lysed in 1×SDS-PAGE buffer and loaded on SDS-12% PAGE.

Assays of Murine Cells. For assays of tissues and cells, mice were sacrificed a week after arrival. Three mice were sacrificed for each strain or model to obtain the results in FIGS. 22 and 28. Serum samples were obtained by tail bleeds and repeated (n=3 per mouse). Splenocytes were filtered through cell strainers (Becton Dickinson), and treated with red cell lysing buffer (Sigma). Splenocytes were counted using a hemochromocytometer, and plated at 2 million cells/ml on plastic 6-well Falcon tissue culture dishes (Becton Dickinson) in RPMI with 10% FCS. Bone marrow-cultures were isolated by flushing femoral and tibial marrow into culture, filtering through cell strainer, followed by red cell lysis, and then plating population on plastic 6-well Falcon tissue culture dishes in 2 ml RPMI containing 10% FBS, 2 mM HEPES, 50 mM b-mercaptoethanol, 20 ng/mL GM-CSF, and 1 ng/mL IL-4. The cells were incubated at 37° C. in a humidified 5% CO₂ atmosphere with media changes and removal of non-adherent cells every 3 days. After 7 days of culture, a homogeneous population of adherent cells was obtained. Splenocytes were lysed for immunoblots either on isolation or after 36 hours in culture; results were similar. Immunoblots for each sample were performed in duplicate. The Student's t-test was used to compare paired means from expression studies, while activity assays for mouse-derived samples were compared using Wilcoxon rank sum tests.

Therapeutic Uses. The experimental data indicates that D1 and D1L3 function as non-overlapping nucleases that protect tissues against exogenous DNA. D1 protects against free DNA, while D1L3 targets membrane-bound DNA.

In Lupus, deficient clearance of apoptotic debris by DNases and complement has been speculated to lead to autoimmunity against nucleosomal antigens. Recently, apoptotic bodies themselves, much like DNA-coated liposomes, have been found to horizontally transduce DNA in vitro. Circulating apoptotic debris or infectious agents, for example, may resemble the liposomal units targeted by D1L3 in these experiments. Accordingly, increasing D1L3 activity by administration of D1L3 or compounds that induce D1L3 activity may provide treatment for Lupus.

The observations also have a number of implications for gene therapy. Initially, D1L3 should be the standard nuclease to evaluate the in vivo potential of transfecting agents. Moreover, blocking this macrophage-secreted activity by administration of agents that prevent expression of this enzyme or inhibit its activity may also enhance gene therapy.

Finally, administration of D1L3 or D1L3 inducing agents may provide treatment against membrane bound pathogenic DNA. Suitable D1L3 inducing agents include, for example, interferon-gamma, LPS, Phorbol-myristate acetate and the like.

1. A gene therapy composition, comprising a recombinant gene to affect the gene therapy, a lipofection reagent, and a D3-activity-reducing agent.

2. The gene therapy composition according to claim 1, wherein the lipofection reagent is selected from the group consisting of monocationic lipids, polycationic lipids, DEAE, dextran, lipo-polyamines, and cholesterol.

3. The gene therapy composition according to claim 2, wherein the D3-activity-reducing agent is selected from the group consisting of antibodies, peptides, DNA fragments, and chemicals.

4. The gene therapy composition according to claim 2, wherein the D3-activity-reducing agent is selected from the group consisting of monoclonal antibodies, enzymatic inhibitors, and enzymes that target D1L3.

5. The gene therapy composition according to claim 4, wherein the D3-activity-reducing agent selectively inhibits D1L3 expression, inhibits D1L3 nuclease activity, inhibits C-terminus activity, complexes D1L3, or degrades D1L3.

6. An antiviral composition comprising an effective amount of D1L3 or a D1L3 inducing agent selected from interferon-gamma or LPS.

7. A chimeric DNase comprising a wild-type DNase attached to a D1L3 C-terminus selected from sequences A, B, and C in FIG. 14.

8. A lipofection efficiency testing composition comprising D1L3.

9. A method of testing lipofection efficiency comprising exposing a transfection composition to a target cell in the presence of D1L3.

10. A method of protecting against, treating, or reversing the progression of lupus in a mammal, said method comprising increasing D1L3 activity in the mammal.

11. The method of claim 10, wherein D1L3 activity is increased by intravenous administration of D1L3 or a D1L3 inducing agent.

12. The method of claim 11, wherein the D1L3 inducing agent is interferon-gamma.

13. A method of destroying a pathogenic encapsulated, membrane bound or micellar bound DNA comprising increasing D1L3 activity.

14. The method of claim 13, wherein D1L3 activity is increased by intravenous administration of D1L3 or a D1L3 inducing agent.

15. The method of claim 14, wherein the D1L3 inducing agent is interferon-gamma.

16. The method of claim 14, wherein the pathogenic encapsulated, membrane bound or micellar bound DNA is selected from the group consisting of infectious, mostly viral particles, oncogenic/mutagenic DNA in apoptotic debris, and intracellular microbacterial pathogens.

17. A method of treating a disease in a mammal by gene therapy, said method comprising the administration of a gene therapy composition to the mammal, wherein the gene therapy composition comprises a recombinant gene to affect the gene therapy, a lipofection reagent, and a D3-activity-reducing agent.

18. The method of treating a disease according to claim 17, wherein the gene therapy targets vascular organs containing large amounts of reticuloendothelial or immunologic cells.

19. The method of treating a disease according to claim 18, wherein the reticuloendothelial or immunologic cells are selected from the group consisting of lymphocytes, monocyte-related cells and monocyte-derived cells.

20. The method of treating a disease according to claim 18, wherein the vascular organs are selected from the group consisting of liver, spleen thymus, bone marrow, lymphoid tissues, lung, pancreas, gut, kidney, and vascular endothelium.

21. The method of treating a disease according to claim 17, wherein the disease is located in a tissue directly bathed by circulation with or without a blood-brain barrier.

22. A method of identifying an individual having a mutation associated with lupus comprising:
a) providing a nucleic acid sample from the individual, wherein the nucleic acid sample comprises a nucleic acid sequence encoding DNASE1L3 or DNASE3; and
b) detecting a mutation in the nucleic acid sequence, wherein the presence of the mutation identifies the individual as having a mutation associated with lupus.


SEQUENCE LISTING (85 sequences)

SEQ ID NO: 1 - protein, 301 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYNDEDDVFAREPFVAQFSLPSNVLPSLVLVPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 2 - protein, 283 aa, Homo sapiens
MGGPRALLAALWALEAAGTAALRIGAFNIQSFGDSKVSDPACGSIIAKIPAGYDLALVQEVRDPDLSAVSALMEQINSVSEHEYSFVSSQPLGRDQYKEMYLFVYRKDAVSVVDTYLYHDYQDGDADVFSREPFVVWFQSPHTAVKDFVIIPLHTTPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 3 - protein, 323 aa, Homo sapiens
MHYPTALLFLILANGAQAFRICAFNAQRLTLAKVAREQVMDTLVRILARCDIMVLQEVVDSSGSAIPLLLREINRFDDSGPYSTLSSPQLGRSTYMETYVYFYRSHKTQVLSSYVYPDPEDVFSREPFVVKFSAPGTGERAPPLPSRRALTPPPLPAAAQNLVLIPLHAAPKAVEKELNALYDVFLEVSQHWQSKDVILLGDFNADCASLTKKRLDKLELRTEPSFHWVIADGEDTTVRASTHCTYDRVVLHGERCRSLLHTAAAFDFPTSFQLLTEEEALNISDHYPVEVELKLSQAHSVQPLSLTVLLLLSLLSPQLCPAA

SEQ ID NO: 4 - protein, 282 aa, Homo sapiens [sequence omitted]

SEQ ID NO: 5 - protein, 34 aa, Mus musculus
SDHFPVEFKLQSSRAFTNNRKSVSLKKRKKGNRS

SEQ ID NO: 6 - protein, 34 aa, Rat
SDHFPVEFKLQSSRAFTNSRKSVSLKKKKKGSRS

SEQ ID NO: 7 - protein, 34 aa, Homo sapiens
SDHFPVEFKLQSSRAFTNSKKSVTLRKKTKSKRS

SEQ ID NO: 8 - protein, 27 aa, Xenopus sp.
SDHFPIEVRLKESKRPTSRRRKYFKRK

SEQ ID NO: 9 - protein, 10 aa, Bovine
SDHYPVEVTL

SEQ ID NO: 10 - protein, 40 aa, Homo sapiens
SDHYPVEVELKLSQAHSVQPLSLTVLLLLSLLSPQLCPAA

SEQ ID NO: 11 - DNA, 998 bp, Murine
tcagtgagccaggcactgtcttcatccagcctgaagtcccaggagtgcaaagatgtccctgcacccagcttccccacgcctggcctccctgctgctcttcatccttgccctccatgacaccctggccctaaggctctgctccttcaatgtgaggtcctttggagcgagcaagaaggaaaaccatgaagccatggatatcattgtgaagatcatcaaacgctgtgaccttatactgttgatggaaatcaaggacagcagcaacaacatctgtcccatgctgatggagaagctgaatggaaattcacgaagaagcacaacatacaactatgtgattagttctcgacttggaagaaacacgtacaaagagcagtatgccttcgtctacaaggagaagctggtgtctgtgaagacaaaataccactaccatgactatcaggatggagacacagacgtgttttccagggagccctttgtggtttggttccattccccctttactgctgtcaaggacttcgtgattgtccccttgcacacaactcccgagacctccgttaaagagatagatgagctggtcgatgtctacacggatgtgagaagccagtggaagacagagaatttcatcttcatgggtgatttcaacgccggctgtagctatgtccccaagaaggcctggcagaacattcgtttgaggacggaccccaagtttgtttggctgattggggaccaagaggacactacggtcaagaagagtaccagctgtgcctatgacaggattgtgctttgtggacaagagatagtcaactccgtggttccccgttccagtggcgtctttgactttcagaaagcttatgacttgtctgaggaggaggccctggatgtcagtgatcactttccagttgagtttaagctacagtcttcaagggccttcaccaacaacagaaaatctgtttctctcaaaaagagaaaaaaaggcaatcgctcctaggtatcacgctccg

SEQ ID NO: 12 - protein, 310 aa, Murine
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRS

SEQ ID NO: 13 - DNA, 26 bp, Artificial
  Mutagenesis Primer
ggtgatttcaaggccggctgtagcta

SEQ ID NO: 14 - protein, 310 aa, Artificial
  N196K-D3 cDNA
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFKAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRS

SEQ ID NO: 15 - DNA, 23 bp, Artificial
  Mutagenesis Primer
agctacagtcttaaagggccttc

SEQ ID NO: 16 - DNA, 31 bp, Murine
gtttaagctacagtcttcaagggccttcacc

SEQ ID NO: 17 - protein, 10 aa, Murine
FKLQSSRAFT

SEQ ID NO: 18 - DNA, 871 bp, Mus musculus
gctttcaggatgcggtacacagggctaatgggaacactgctcaccttggtcaacctgctgcagctggctgggactctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaaaatctgatgtcatt

SEQ ID NO: 19 - protein, 303 aa, Artificial
  Fusion Protein
YTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVVRALLQAAVVPNSAVPFDFQAEYGLSNQLAEAISDHYPVEVTLRKIRAFTNNRKSVSLKKRKKGNRS

SEQ ID NO: 20 - DNA, 967 bp, Artificial
  Fusion Clone
ctgctgcagccgtctcagattggctttcaggatgcggtacacagggctaatgggaacactctcaccttggtcaacctgctgcagctggctgggactctgagaattgcagccttcaacattcggacttttggggagactaagatgtccaatgctaccctctctgtatactttgtgaaaatcctgagtcgctatgacatcgctgttatccaagaggtcagagactcccacctggttgctgttgggaagctcctggatgaactcaatcgggacaaacctgacacctaccgctatgtagtcagtgagccgctgggccgcaaaagctacaaggaacagtacctttttgtgtacaggcctgaccaggtgtctattctggacagctatcaatatgatgatggctgtgaaccctgtggaaatgacaccttcagcagagagccagccattgttaagttcttttccccatacactgaggtccaagaatttgcgatcgtgcccttgcatgcagccccaacagaagctgtgagtgagatcgacgccctctacgatgtttacctagatgtctggcaaaagtggggcctggaggacatcatgttcatgggagacttcaatgctggctgcagctacgtcacttcctcccagtggtcctccattcgccttcggacaagccccatcttccagtggctgatccctgacagtgcggacaccacagtcacatcaacacactgtgcttatgacaggattgtggttgctggagctctgctccaggctgctgttgttcccaactcggctgttccttttgatttccaagcagaatacggactttccaaccagctggctgaagccatcagtgaccattacccagtggaggtgacactcagaaagatccgggccttcaccaacaacagaaaatctgtttctctcaaaaagagaaaaaaaggcaatcgctcctaggtatcacgctccggaattc

SEQ ID NO: 21 - DNA, 25 bp, Artificial
  Mutagenesis Primer
cactcagaaagatctgatgtcattg

SEQ ID NO: 22 - DNA, 29 bp, Artificial
  Primer
gcggatccgggccttcaccaacaacagaa

SEQ ID NO: 23 - protein, 59 aa, Murine
LRIAAFNIRTFGETKMSNATLSVYFVKILSRYDIAVIQEVRDSHLVAVGKLLDELNRDK

SEQ ID NO: 24 - protein, 59 aa, Bovine
LKIAAFNIRTFGETKMSNATLASYIVRIVRRYDIVLIQEVRDSHLVAVGKLLDYLNQDD

SEQ ID NO: 25 - protein, 51 aa, Murine
YVVSEPLGRKSYKEQYLFVYRPDQVSILDSYQYDDGCEPCGNDTFSREPAI

SEQ ID NO: 26 - protein, 51 aa, Bovine
YVVSEPLGRNSYKERYLFLFRPNKVSVLDTYQYDDGCESCGNDSFSREPAV

SEQ ID NO: 27 - DNA, 24 bp, Artificial
  Primer
actaagatgtccagtgctaccctc

SEQ ID NO: 28 - DNA, 24 bp, Artificial
  Primer
gagggtagcactggacatcttagt

SEQ ID NO: 29 - DNA, 24 bp, Artificial
  Primer
gaaccctgtggaagtgacaccttc

SEQ ID NO: 30 - DNA, 24 bp, Artificial
  Primer
gaaggtgtcacttccacagggttc

SEQ ID NO: 31 - DNA, 18 bp, Murine
cacaatatacaactatct

SEQ ID NO: 32 - DNA, 18 bp, Murine
cacaacatacaactatgt

SEQ ID NO: 33 - DNA, 21 bp, Murine
cgaagaagcacaatatacaac

SEQ ID NO: 34 - protein, 7 aa, Murine
RRSTIYN

SEQ ID NO: 35 - protein, 20 aa, Mouse
GNSRRRSTTYNYVISSRLGR

SEQ ID NO: 36 - protein, 20 aa, Mouse
GNSRRRSTIYNYVISSRLGR

SEQ ID NO: 37 - protein, 20 aa, Rat
GNSRRRSTTYNYVISSRLGR

SEQ ID NO: 38 - protein, 20 aa, Homo sapiens
RNSRRRGITYNYVISSRLGR

SEQ ID NO: 39 - protein, 16 aa, Homo sapiens
DAPDTYHYVVSEPLGR

SEQ ID NO: 40 - DNA, 23 bp, Artificial
  Primer
aatggaaattcacgaagaagcac

SEQ ID NO: 41 - DNA, 24 bp, Artificial
  Primer
cgagaactaatcacatagttgaat

SEQ ID NO: 42 - DNA, 14 bp, Homo sapiens
aagcttacaagctg

SEQ ID NO: 43 - DNA, 14 bp, Homo sapiens
aagcttgcaagctg

SEQ ID NO: 44 - DNA, 14 bp, Homo sapiens
aagcttgcaagctg

SEQ ID NO: 45 - DNA, 14 bp, Homo sapiens
aagcttacaagctg

SEQ ID NO: 46 - protein, 16 aa, Mouse
FQKAYDLSEEEALDVS

SEQ ID NO: 47 - protein, 16 aa, Rat
FQKAYELSEEEALDVS

SEQ ID NO: 48 - protein, 16 aa, Xenopus sp.
FQKAYKLTEEEALDVS

SEQ ID NO: 49 - protein, 16 aa, Homo sapiens
FMVAYGLTEEQALEVS

SEQ ID NO: 50 - protein, 16 aa, Homo sapiens
FQAAYGLSDQLAQAIS

SEQ ID NO: 51 - protein, 16 aa, Homo sapiens
FQEEFGLDQTQALAIS

SEQ ID NO: 52 - DNA, 12 bp, Homo sapiens
gaggtgaggact

SEQ ID NO: 53 - DNA, 12 bp, Homo sapiens
gaggtgakgact

SEQ ID NO: 54 - DNA, 12 bp, Homo sapiens
gaggtgatgact

SEQ ID NO: 55 - DNA, 22 bp, Artificial
  Primer
cagcactccaagcactgctgtc

SEQ ID NO: 56 - DNA, 22 bp, Artificial
  Primer
aagtctgcagacaggagagagg

SEQ ID NO: 57 - DNA, 22 bp, Artificial
  Primer
tctgtggtaaatgacctcaaac

SEQ ID NO: 58 - DNA, 22 bp, Artificial
  Primer
tgtgtgaactggtggtcaagtg

SEQ ID NO: 59 - DNA, 25 bp, Artificial
  Primer
gaatgtttactcttcaagatgcagt

SEQ ID NO: 60 - DNA, 23 bp, Artificial
  Primer
cgatttattggtggccatgttcc

SEQ ID NO: 61 - DNA, 21 bp, Artificial
  Primer
tactcttcaagatgcagtgtt

SEQ ID NO: 62 - DNA, 20 bp, Artificial
  Primer
attggtggccatgttccagg

SEQ ID NO: 63 - DNA, 22 bp, Artificial
  Primer
gctgactcagctgggaggactt

SEQ ID NO: 64 - DNA, 23 bp, Artificial
  Primer
ttaatgggctcatgctcaaggct

SEQ ID NO: 65 - DNA, 23 bp, Artificial
  Primer
agtggtttatgagaccttgacag

SEQ ID NO: 66 - DNA, 23 bp, Artificial
  Primer
tgctagatgggaattcgtctgac

SEQ ID NO: 67 - DNA, 23 bp, Artificial
  Primer
cagtgtgaaagcattctgcagca

SEQ ID NO: 68 - DNA, 22 bp, Artificial
  Primer
gcacaccaagcactgtggtgag

SEQ ID NO: 69 - DNA, 23 bp, Artificial
  Primer
cttggtttgatctcagcaatcac

SEQ ID NO: 70 - DNA, 23 bp, Artificial
  Primer
atctttccaggacaatggcatag

SEQ ID NO: 71 - DNA, 23 bp, Artificial
  Primer
ggcatctcctaatttccatgtct

SEQ ID NO: 72 - DNA, 23 bp, Artificial
  Primer
ccttccaatttggctcaagtcag

SEQ ID NO: 73 - DNA, 21 bp, Artificial
  Primer
gcactgtcttcatccagcctg

SEQ ID NO: 74 - DNA, 22 bp, Artificial
  Primer
cttaaggcctcgcactctggat

SEQ ID NO: 75 - DNA, 20 bp, Artificial
  Primer
ccaccactgcaaagatgtcc

SEQ ID NO: 76 - DNA, 21 bp, Artificial
  Primer
cttctgacatcgaatttgagt

SEQ ID NO: 77 - DNA, 22 bp, Artificial
  Primer
ctgctgcagccgtctcagattg

SEQ ID NO: 78 - DNA, 22 bp, Artificial
  Primer
aagcagtatggctgaactgctc

SEQ ID NO: 79 - DNA, 24 bp, Artificial
  Primer
gcagagctggtttagtgaaccgtc

SEQ ID NO: 80 - DNA, 24 bp, Artificial
  Primer
gagcgtggtacctaggagcgattg

SEQ ID NO: 81 - DNA, 22 bp, Artificial
  Primer
ttcgtcataccgacttgacgag

SEQ ID NO: 82 - DNA, 23 bp, Artificial
  Mutagenesis Primer
agctacagtcttaaagggccttc

SEQ ID NO: 83 - protein, 12 aa, Homo sapiens
KAYDLSEEEALD

SEQ ID NO: 84 - DNA, 22 bp, Artificial
  Primer
ctgctgcagccgtctcagattg

SEQ ID NO: 85 - DNA, 22 bp, Artificial
  Primer
gagcagttcagccatactgctt